(19) United States
(12) Patent Application Publication
(10) Pub. No.: US 2025/0075006 A1
(43) Pub. Date: Mar. 6, 2025

(54) ANTI-GUCY2C NANO-ANTIBODY AND APPLICATION THEREOF

(71) Applicant: Shandong Simcere Biopharmaceutical Co., Ltd, Yantai, Shandong (CN)

(72) Inventors: Guangcun Cheng, Shanghai (CN); Yayuan Fu, Shanghai (CN); Zhuoxiao Cao, Shanghai (CN); Ling Li, Shanghai (CN); Renhong Tang, Shanghai (CN); Jinsheng Ren, Nanjing, Jiangsu (CN)

(73) Assignee: Shandong Simcere Biopharmaceutical Co., Ltd., Yantai, Shandong (CN)

(21) Appl. No.: 18/722,098

(22) PCT Filed: Dec. 22, 2022
(86) PCT No.: PCT/CN2022/140883

§ 371 Date: Jun. 20, 2024

(30) Foreign Application Priority Data
Dec. 23, 2021 (CN) 202111589520.7

(51) Int. Cl. C07K 16/40; C12N 5/0783; G01N 33/573

(52) U.S. Cl. CPC C07K 16/40; C12N 5/0636; C12Y 406/01002; G01N 33/573; C07K 2317/24; C07K 2317/53; C07K 2317/569; C07K 2317/92; C12N 2510/00; G01N 2333/988

(57) ABSTRACT

An antibody capable of specifically binding to GUCY2C or an antigen-binding fragment thereof. The antibody or the antigen-binding fragment thereof can specifically bind to GUCY2C with high affinity, and can be taken as a drug to treat gastrointestinal malignancies.

### CROSS-REFERENCE TO RELATED PATENT APPLICATION

The present application claims priority to the Chinese Patent Application No. 202111589520.7 filed with China National Intellectual Property Administration on Dec. 23, 2021, and entitled “ANTI-GUCY2C NANO-ANTIBODY AND APPLICATION THEREOF”, which is incorporated herein by reference in its entirety.

### TECHNICAL FIELD

The present application relates to the field of antibodies, in particular to an anti-GUCY2C nanobody.

### BACKGROUND

Malignant gastrointestinal tumors include colorectal cancer (CRC), gastric cancer, and esophageal cancer. Colorectal cancer, also known as large intestine cancer, is a common malignant tumor of the digestive system. At present, the treatment for CRC is mainly surgery and chemoradiotherapy. Patients with early stage CRC can receive surgery or/and chemotherapy, but there's no effective treatment for patients with advanced CRC and patients with metastatic colorectal cancer in clinical application. In recent years, immune checkpoint inhibitors, as represented by PD1/L1, have been developed in a variety of tumor treatments, but the treatment for colorectal cancer is still challenging. Most colorectal cancer patients are of the microsatellite stability type, which has a low tumor mutation burden and a reduced immune infiltration density, and is often accompanied by KRAS or BRAF oncogene mutation, resulting in colorectal cancer patients' lack of response to currently approved immunotherapies, which fails to demonstrate significant survival benefits. Guanylate cyclase C (GUCY2C or GCC) is a target that is widely expressed in colorectal cancer and other gastrointestinal tumors. In normal tissues, GUCY2C plays an important role in maintaining intestinal fluid and electrolyte balance and cell proliferation. The intracellular enzyme catalytic region is capable of binding to GTP, catalyzing the conversion of GTP to cGMP, and generating a second messenger affecting the downstream signaling pathway. GUCY2C is expressed only at mucosal cells of the small intestine, large intestine, and rectum, while it is expressed in all primary and metastatic colorectal tumors. Therefore, GUCY2C has become an attractive target, and the development of drugs targeting GUCY2C is of great clinical significance for treatment of colorectal cancer.

### SUMMARY

The present application provides the following embodiments.

In one aspect, the present application provides a nanobody or an antigen-binding fragment thereof specifically binding to GUCY2C, wherein the antibody or the antigen-binding fragment thereof comprises an HCDR1, an HCDR2, and an HCDR3, the HCDR1 comprises an HCDR1 of the VH set forth in any one of SEQ ID NOs: 14-16, 47-49, and 51-63, the HCDR2 comprises an HCDR2 of the VH set forth in any one of SEQ ID NOs: 14-16, 47-49, and 51-63, and the HCDR3 comprises an HCDR3 of the VH set forth in any one of SEQ ID NOs: 14-16, 47-49, and 51-63.

In another aspect, the present application provides a multispecific molecule, wherein the multispecific molecule comprises any one of the foregoing nanobodies or the antigen-binding fragment thereof.

In another aspect, the present application provides a chimeric antigen receptor (CAR), wherein the chimeric antigen receptor at least comprises an extracellular antigen-binding domain, a transmembrane domain, and an intracellular signaling domain; the extracellular antigen-binding domain comprises any one of the foregoing nanobodies or the antigen-binding fragment thereof.

In another aspect, the present application provides an immune effector cell, wherein the immune effector cell expresses the foregoing chimeric antigen receptor or comprises a nucleic acid fragment encoding the chimeric antigen receptor.

In another aspect, the present application provides an isolated nucleic acid fragment, wherein the nucleic acid fragment encodes any one of the foregoing nanobodies or the antigen-binding fragment thereof, or the multispecific molecule, or the chimeric antigen receptor.

In another aspect, the present application provides a vector, wherein the vector comprises the nucleic acid fragment.

In another aspect, the present application provides a host cell, wherein the host cell comprises the vector.

In another aspect, the present application provides a method for preparing any one of the foregoing nanobodies or the antigen-binding fragment thereof, or multispecific molecules, wherein the method comprises culturing the host cell, and isolating a nanobody or an antigen-binding fragment thereof expressed by the cell, or isolating a multispecific molecule expressed by the cell.

In another aspect, the present application provides a method for preparing the immune effector cell, wherein the method comprises introducing a nucleic acid fragment encoding the CAR into the immune effector cell.

In another aspect, the present application provides a pharmaceutical composition, wherein the pharmaceutical composition comprises any one of the foregoing nanobodies or the antigen-binding fragment thereof, multispecific antibodies, immune effector cells, nucleic acid fragments, vectors, or host cells, or a product prepared by any one of the foregoing methods.

In another aspect, further provided is use of any one of the foregoing nanobodies or the antigen-binding fragment thereof, multispecific molecules, immune effector cells, nucleic acid fragments, vectors, host cells, products prepared by any one of the foregoing methods, or pharmaceutical compositions disclosed herein in preparing a medicament for preventing and/or treating a tumor, wherein the tumor may be selected from colorectal cancer, gastric cancer, small intestine cancer, esophageal cancer, pancreatic cancer, lung cancer, soft tissue sarcoma, and neuroendocrine tumor.

In another aspect, the present application provides a method for preventing and/or treating a tumor, wherein the method comprises administering to a patient in need thereof an effective amount of any one of the foregoing nanobodies or the antigen-binding fragment thereof, multispecific molecules, immune effector cells, nucleic acid fragments, vectors, host cells, products prepared by any one of the foregoing methods, or pharmaceutical compositions, wherein the tumor is selected from colorectal cancer, gastric cancer, small intestine cancer, esophageal cancer, pancreatic cancer, lung cancer, soft tissue sarcoma, and neuroendocrine tumor.

In another aspect, the present application further provides any one of the foregoing nanobodies or the antigen-binding fragment thereof, multispecific molecules, immune effector cells, nucleic acid fragments, vectors, host cells, products prepared by any one of the foregoing methods, or pharmaceutical compositions for use in preventing and/or treating a tumor, wherein the tumor is selected from colorectal cancer, gastric cancer, small intestine cancer, esophageal cancer, pancreatic cancer, lung cancer, soft tissue sarcoma, and neuroendocrine tumor.

In another aspect, the present application provides a kit, wherein the kit comprises any one of the foregoing nanobodies or the antigen-binding fragment thereof, multispecific antibodies, immune effector cells, nucleic acid fragments, vectors, host cells, products prepared by any one of the foregoing methods, or pharmaceutical compositions.

In another aspect, the present application provides a method for detecting GUCY2C expression, comprising contacting a sample to be tested with any one of the foregoing nanobodies or the antigen-binding fragment thereof in a condition allowing formation of a complex by any one of the foregoing nanobodies or the antigen-binding fragment thereof and GUCY2C.

In another aspect, the present application provides a method for inhibiting the proliferation or migration of a cell expressing GUCY2C in vitro, comprising contacting the cell with any one of the foregoing nanobodies or the antigen-binding fragment thereof in a condition allowing formation of a complex by any one of the foregoing nanobodies or the antigen-binding fragment thereof and GUCY2C.

The nanobody or the antigen-binding fragment thereof provided by the present application can specifically bind to GUCY2C at protein and cell levels, has good affinity with human and monkey GUCY2C proteins, and provides an excellent choice for developing medicines targeting GUCY2C.

### BRIEF DESCRIPTION OF THE DRAWING

FIG. 1 shows expression levels of 293T-hGUCY2C and CHO-cyno GUCY2C cell lines as assayed by flow cytometry.

A and B in FIG. 2 are electrophoresis diagrams of the first round and the second round of amplifications of Vicugna pacos nanobody gene sequences by nested PCR, wherein the lane M is a protein Marker band.

FIG. 3 shows the number of transformants in a plate, wherein the left plate is the number of transformants in the bacterium solution diluted 1000-fold and the right plate is the number of transformants diluted 10000-fold.

FIG. 4 is an electrophoresis diagram for detecting the insertion rate of the clones after electroporation by PCR, wherein the lane M is a protein Marker band.

FIG. 5 is a spectrogram of assay on purity of the Vicugna pacos VHH-huFc antibodies of the present application by the SEC-HPLC method.

FIG. 6 shows binding reactions of VHH recombinant antibodies with a human GUCY2C-his protein by ELISA.

FIG. 7 shows binding reactions of VHH recombinant antibodies with a monkey GUCY2C-his protein by ELISA.

FIG. 8 shows binding reactions of VHH recombinant antibodies with a murine GUCY2C-his protein by ELISA.

FIG. 9 shows binding reactions of VHH recombinant antibodies with 293T-hGUCY2C cells as assayed by FACS.

FIG. 10 shows binding reactions of VHH recombinant antibodies with endogenous tumor cells HT-55 as assayed by FACS.

FIG. 11 shows binding reactions of VHH recombinant antibodies with CHO-cyno GUCY2C cells as assayed by FACS.

A-C in FIG. 12 shows binding reactions of humanized antibodies Lab306, Lab322, and Lab323 with a human GUCY2C-his protein by ELISA.

A-C in FIG. 13 shows binding reactions of humanized antibodies Lab306, Lab322, and Lab323 with a monkey GUCY2C-his protein by ELISA.

A-C in FIG. 14 shows binding reactions of humanized antibodies with human 293T-hGUCY2C cells as assayed by FACS.

A-C in FIG. 15 shows binding reactions of humanized antibodies with endogenous tumor cells HT-55 as assayed by FACS.

A-C in FIG. 16 shows binding reactions of humanized antibodies with CHO-cyno GUCY2C cells as assayed by FACS.

FIG. 17 shows the epitope difference between antibodies as assayed by competitive ELISA.

### DETAILED DESCRIPTION OF THE INVENTION

### Terminology and Definitions

Unless otherwise defined herein, scientific and technical terms used in correlation with the present application shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “including”, “comprising”, and “having” herein are used interchangeably and are intended to indicate the inclusion of a solution, implying that there may be elements other than those listed in the solution. Meanwhile, it should be understood that the descriptions “including”, “comprising”, and “having” as used herein also provide the solution of “consisting of . . . ”. Illustratively, “a composition, comprising A and B” should be understood as the following technical solution: a composition consisting of A and B, and a composition containing other components in addition to A and B, all fall within the scope of the aforementioned “a composition”.

The term “and/or” as used herein includes the meanings of “and”, “or”, and “all or any other combination of elements linked by the term”.

The term “GUCY2c” herein refers to mammalian guanylate cyclase C (GUCY2C), preferably human GUCY2C protein. The term “GUCY2C” may be used interchangeably with “STAR”, “GUC2C”, “GCC”, or “ST receptor”. The nucleotide sequence of human GUCY2C is disclosed as GenBank accession No. NM_004963, and the amino acid sequence of human GUCY2C is disclosed as GenBank accession No. NP_004954. Typically, naturally occurring allelic variants have an amino acid sequence that is at least 95%, 97%, or 99% identical to the protein described in GenBank accession No. NP.Sub.-004954. GUCY2C protein is a transmembrane cell surface receptor protein, and plays an important role in maintaining intestinal fluid and electrolyte homeostasis, cell proliferation, and the like.

The term “specific binding” herein means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), where a relatively low KD indicates a relatively high affinity. In the case of antibodies, high affinity generally means having a KD of about 1×10⁻⁶ M or less, 1×10⁻⁷ M or less, about 1×10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis. Illustratively, KD can be obtained by the method as described in Example 4 or 7 herein.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Exemplarily, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen-binding activity. “Antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Komdorfer et al., 2003, Proteins: Structure, Function, and Bioinformatics, 53(1): 121-129 (2003); and Roque et al., Biotechnol. Prog. 20: 639-654 (2004). Such scaffolds may also include non-antibody derived scaffolds, such as scaffold proteins known in the art to be useful for grafting CDRs, including, but not limited to tenascin, fibronectin, peptide aptamers, and the like. “Antibody” herein includes antibodies that do not comprise a light chain, e.g., heavy chain antibodies (HCAbs) produced by Camelidae species such as Camelus dromedarius, Camelus bactrianus, Lama glama, Lama guanicoe, and Vicugna pacos, as well as immunoglobulin new antigen receptors (IgNARs) found in Chondrichthyes, e.g., shark.

As used herein, the term “heavy chain antibody” refers to an antibody lacking a light chain of a conventional antibody. The term specifically includes, but is not limited to, homodimeric antibodies comprising a VH antigen-binding domain and CH2 and CH3 constant domains in the absence of a CH1 domain.

As used herein, the term “nanobody” refers to a heavy chain antibody naturally lacking a light chain present in a camel, and the cloning of its variable region can give a single domain antibody only consisting of a heavy chain variable region (also called VHH (variable domain of heavy chain of heavy chain antibody)), which is the smallest functional antigen-binding fragment.

The terms “nanobody” and “single domain antibody (sdAb)” herein have the same meaning and can be used interchangeably, and refer to a single domain antibody consisting of only one heavy chain variable region constructed by cloning a variable region of a heavy chain antibody, which is the smallest antigen-binding fragment having the complete function. Generally, a single domain antibody consisting of only one heavy chain variable region is constructed by obtaining a heavy chain antibody naturally lacking a light chain and a heavy chain constant region 1 (CH1) and then cloning a variable region of an antibody heavy chain.

For further description of “heavy chain antibody” and “nanobody”, see: Hamers-Casterman et al., Nature. 1993; 363; 446-8; a review article (Reviews in Molecular Biotechnology 74: 277-302, 2001) by Muyldermans; and the following patent applications mentioned as general background art: WO 94/04678, WO 95/04079, and WO 96/34103; WO94/25591, WO 99/37681, WO 00/40968, WO 00/43507, WO 00/65057, WO 01/40310, WO 01/44301, EP 1134231, and WO 02/48193; WO97/49805, WO 01/21817, WO 03/035694, WO 03/054016, and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787, and WO 06/122825, as well as other prior art mentioned in these applications. “Antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “multispecific” herein means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule. “Antigen-binding fragment” and “antibody fragment” herein are used interchangeably and do not have the entire structure of an intact antibody, but comprise only a portion of the intact antibody or a variant of the portion that has the ability to bind to an antigen. “Antigen-binding fragment” or “antibody fragment” herein includes but is not limited to, a Fab, a Fab′, a Fab′-SH, a F(ab′)₂, an Fd, an Fv, an scFv, a diabody, and a single domain antibody.

The term “chimeric antibody” herein refers to an antibody in which a portion of the light chain or/and heavy chain is derived from one antibody (which may be derived from a particular species or belong to a particular antibody class or subclass) and another portion of the light chain or/and heavy chain is derived from another antibody (which may be derived from the same or a different species or belong to the same or a different antibody class or subclass), but which nevertheless retains binding activity to a target antigen (Cabilly et al., U.S. Pat. No. 4,816,567; Morrison et al., Proc. Natl. Acad. Sci. USA, 81: 6851 6855(1984)). For example, the term “chimeric antibody” can include an antibody (e.g., a human-murine chimeric antibody) in which the heavy and light chain variable regions of the antibody are derived from a first antibody (e.g., a murine antibody) and the heavy and light chain constant regions of the antibody are derived from a second antibody (e.g., a human antibody).

The term “humanized antibody” herein refers to a genetically engineered non-human antibody that has an amino acid sequence modified to increase homology to the sequence of a human antibody. Generally, all or part of the CDRs of a humanized antibody is derived from a non-human antibody (donor antibody), and all or part of the non-CDRs (e.g., variable region FRs and/or constant regions) is derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See, e.g., Kindt et al., Kuby Immunology, 6th ed., W. H. Freeman and Co., p.91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it is precisely complementary to an epitope in a spatial structure, wherein the heavy chain variable region CDR may be abbreviated as HCDR and the light chain variable region CDR may be abbreviated as LCDR. The terms “framework region” or “FR” are used interchangeably and refer to those amino acid residues of an antibody heavy chain variable region or light chain variable region, other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J Biol. Chem., 252: 6609-6616 (1977); Kabat et al., United States Department of Health and Human Services, Sequences of proteins of immunological interest (1991); Chothia et al., J Mol. Biol. 196: 901-917 (1987); Al-Lazikani B. et al., J Mol. Biol., 273: 927-948 (1997); MacCallum et al., J Mol. Biol. 262: 732-745 (1996); Abhinandan and Martin, Mol. Immunol., 45: 3832-3839 (2008); Lefranc M.P. et al., Dev Comp. Immunol., 27: 55-77 (2003); and Honegger and Pluckthun, J Mol. Biol., 309: 657-670 (2001). “CDR” herein may be labeled and defined in a manner well known in the art, including, but not limited to, Kabat numbering scheme, Chothia numbering scheme, or IMGT numbering scheme; the tool sites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi #results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J Mol. Biol. 196: 901-917; Chothia et al., (1989) Nature 342: 878-883).

The term “Fc” herein refers to the carboxyl-terminal portion of an antibody that is formed by the hydrolysis of an intact antibody by papain, which typically comprises the CH3 and CH2 domains of the antibody. The Fc region includes, for example, an Fc region of native sequences, a recombinant Fc region, and a variant Fc region. Although the boundaries of the Fc region of an immunoglobulin heavy chain may vary slightly, the human IgG heavy chain Fc region is generally defined to stretch from an amino acid residue at position Cys226, or from Pro230, to the carboxyl terminus thereof. The C-terminal lysine of the Fc region (residue 447 according to the Kabat numbering scheme) may be removed, for example, during production or purification of the antibody, or by recombinant engineering of the nucleic acid encoding the heavy chain of the antibody, and thus, the Fc region may or may not include Lys447.

The term “conservative amino acid” herein generally refers to amino acids that belong to the same class or have similar characteristics (e.g., charge, side chain size, hydrophobicity, hydrophilicity, backbone conformation, and rigidity). Illustratively, the amino acids in each of the following groups belong to conservative amino acid residues of each other, and substitutions of amino acid residues within the groups belong to conservative amino acid substitutions: Illustratively, the following six groups are examples of amino acids that are considered to be conservative replacements of each other:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” herein can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48: 444-453; available at www.gcg.com) which has been integrated into the GAP program of the GCG software package, using the Blossum 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum 62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4: 11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or related sequences. For example, such searches can be performed using the NBLAST and XBLAST programs (version 2.0) of Altschul et al., (1990) J. Mol. Biol., 215: 403-10. BLAST nucleotide searches can be performed using the NBLAST program, with a score of 100 and a word length of 12, to obtain nucleotide sequences homologous to the nucleic acid (SEQ ID NO: 1) molecule of the present application. BLAST protein searches can be performed using the XBLAST program, with a score of 50 and a word length of 3, to obtain amino acid sequences homologous to the protein molecule of the present application. To obtain gapped alignment results for the purpose of comparison, gapped BLAST can be used as described in Altschul et al. (1997) Nucleic Acids Res. 25: 3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to be expressed on an immune effector cell and specifically bound to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. In this context, the term “nucleic acid molecule” encompasses deoxyribonucleic acid (DNA), including, e.g., complementary DNA (cDNA) and genomic DNA; ribonucleic acid (RNA), in particular messenger RNA (mRNA); the synthetic forms of DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms.

Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present application in vitro and/or in vivo, e.g., in a host or patient. Such DNA (e.g., cDNA) or RNA (e.g., mRNA) vectors may be unmodified or modified. For example, mRNA may be chemically modified to enhance the stability of the RNA vector and/or the expression of the encoded molecule, so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). “Isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing or slowing (reducing) the progression of an undesired physiological or pathological change, e.g., a cancer, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “subject” herein refers to an organism that receives treatment for a particular disease or disorder described herein. Examples of subjects and patients include mammals, such as humans, primates (e.g., monkey), or non-primate mammals, that receive treatment for a disease or disorder.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues.

The terms “cancer” and “tumor” are not mutually exclusive when referred to herein. The term “EC50” herein refers to the half maximum effective concentration, which includes the antibody concentration that induces a halfway response between the baseline and maximum after a specified exposure time. EC50 essentially represents the antibody concentration at which 50% of the maximal effect is observed, and can be measured by methods known in the art.

As used herein, the term “about” refers to all values within +10% of the specified numerical value.

For example, about 10 may refer to all values in the range of 9-11.

### DETAILED DESCRIPTION

The present application provides an anti-GUCY2C antibody, a nucleic acid for encoding the antibody, an antibody preparation method, a pharmaceutical composition comprising the antibody, and related use of the pharmaceutical composition in treating a tumor.

In one aspect, the present application provides a nanobody or an antigen-binding fragment thereof specifically binding to GUCY2C, wherein the antibody or the antigen-binding fragment thereof comprises an HCDR1, an HCDR2, and an HCDR3, the HCDR1 comprises an HCDR1 of the VH set forth in any one of SEQ ID NOs: 14-16, 47-49, and 51-63, the HCDR2 comprises an HCDR2 of the VH set forth in any one of SEQ ID NOs: 14-16, 47-49, and 51-63, and the HCDR3 comprises an HCDR3 of the VH set forth in any one of SEQ ID NOs: 14-16, 47-49, and 51-63. In some embodiments, the HCDR1, the HCDR2, and the HCDR3 are determined according to the Kabat numbering scheme, the Chothia numbering scheme, or the IMGT numbering scheme.

For example, the HCDR1 comprises the amino acid sequence set forth in SEQ ID NO: 17, 20, 23, 26, 29, 32, 35, 38, or 41, the HCDR2 comprises the amino acid sequence set forth in SEQ ID NO: 18, 21, 24, 27, 30, 33, 36, 39, or 42, and the HCDR3 comprises the amino acid sequence set forth in SEQ ID NO: 19, 22, 25, 28, 31, 34, 37, 40, or 43.

In some embodiments, an HCDR1, an HCDR2, and an HCDR3 of the VH set forth in SEQ ID NO: 14, 47, 51, 52, 53, or 54 are according to the IMGT, Kabat, or Chothia numbering scheme, and have the amino acid sequences set forth in SEQ ID NOs: 17-19, SEQ ID NOs: 26-28, or SEQ ID NOs: 35-37.

In some embodiments, an HCDR1, an HCDR2, and an HCDR3 of the VH set forth in SEQ ID NO: 15, 48, 55, 56, 57, or 58 are according to the IMGT, Kabat, or Chothia numbering scheme, and have the amino acid sequences set forth in SEQ ID NOs: 20-22, SEQ ID NOs: 29-31, or SEQ ID NOs: 38-40.

In some embodiments, an HCDR1, an HCDR2, and an HCDR3 of the VH set forth in SEQ ID NO: 16, 49, 59, 60, 61, 62, or 63 are according to the IMGT, Kabat, or Chothia numbering scheme, and have the amino acid sequences set forth in SEQ ID NOs: 23-25, SEQ ID NOs: 32-34, or SEQ ID NOs: 41-43.

In some embodiments, the nanobody or the antigen-binding fragment thereof comprises CDR sequences having at least 80% identity to the HCDR1, the HCDR2, and the HCDR3 or having 1, 2, 3, or more amino acid insertions, deletions, and/or substitutions compared with the HCDR1, the HCDR2, and the HCDR3, and preferably, the substitutions are conservative amino acid substitutions.

In some embodiments, the nanobody or the antigen-binding fragment thereof comprises the VH set forth in any one of SEQ ID NOs: 14-16, 47-49, and 51-63, or a sequence having at least 80% identity to the VH set forth in any one of SEQ ID NOs: 14-16, 47-49, and 51-63 or at most 20, 19, 18, 17, 16, 15, 14, 13, 12, 11, 10, 9, 8, 7, 6, 5, 4, 3, 2, or 1 mutation compared with the VH set forth in any one of SEQ ID NOs: 14-16, 47-49, and 51-63; the mutation may be selected from an insertion, a deletion, and/or a substitution, and preferably, the substitution is a conservative amino acid substitution.

In some embodiments, the nanobody or the antigen-binding fragment thereof comprises a framework region sequence at least having a mutation, compared with a framework region of the VH set forth in SEQ ID NO: 47, selected from the group consisting of: numbered in the natural order, mutations at positions A24, V29, S30, V37, G44, L45, W47, S49, S74, Q81, and R97.

Preferably, in some embodiments, the nanobody or the antigen-binding fragment thereof comprises a framework region sequence at least having a mutation, compared with a framework region of the VH set forth in SEQ ID NO: 47, selected from the group consisting of: numbered in the natural order, A24T, V29L, S30D, V37F, G44E, L45R, W47G, S49I, S74A, Q81R, and R97A.

In a preferred embodiment, the nanobody or the antigen-binding fragment thereof at least comprises V29L, S30D, V37F, G44E, L45R, W47G, S49I, and R97A mutations compared with a framework region of the VH set forth in SEQ ID NO: 47.

In a preferred embodiment, the nanobody or the antigen-binding fragment thereof at least comprises A24T, V29L, S30D, V37F, G44E, L45R, W47G, S49I, and R97A mutations compared with a framework region of the VH set forth in SEQ ID NO: 47.

In a preferred embodiment, the nanobody or the antigen-binding fragment thereof at least comprises V29L, S30D, V37F, G44E, L45R, W47G, S49I, S74A, and R97A mutations compared with a framework region of the VH set forth in SEQ ID NO: 47.

In a preferred embodiment, the nanobody or the antigen-binding fragment thereof at least comprises V29L, S30D, V37F, G44E, L45R, W47G, S49I, Q81R, and R97A mutations compared with a framework region of the VH set forth in SEQ ID NO: 47.

In some embodiments, the nanobody or the antigen-binding fragment thereof comprises a framework region sequence at least having a mutation, compared with a framework region of the VH set forth in SEQ ID NO: 48, selected from the group consisting of: numbered in the natural order, mutations at positions E1, V2, G26, F27, T28, F29, V37, G44, L45, W47, N74, N77, L79, R87, A88, L93, K98, and M124. Preferably, in some embodiments, the nanobody or the antigen-binding fragment thereof comprises a framework region sequence at least having a mutation, compared with a framework region of the VH set forth in SEQ ID NO: 48, selected from the group consisting of: numbered in the natural order, E1Q, V2L, G26V, F27L, T28N, F29L, V37F, G44E, L45R, W47G, N74R, N77K, L79A, R87K, A88P, L93T, K98V, and M124Q.

In a preferred embodiment, the nanobody or the antigen-binding fragment thereof at least comprises G26V, F27L, T28N, F29L, V37F, G44E, L45R, W47G, N74R, N77K, L79A, and K98V mutations compared with a framework region of the VH set forth in SEQ ID NO: 48.

Preferably, in a preferred embodiment, the nanobody or the antigen-binding fragment thereof at least comprises V2L, G26V, F27L, T28N, F29L, V37F, G44E, L45R, W47G, N74R, N77K, L79A, K98V, and E1Q mutations compared with a framework region of the VH set forth in SEQ ID NO: 48.

In a preferred embodiment, the nanobody or the antigen-binding fragment thereof at least comprises G26V, F27L, T28N, F29L, V37F, G44E, L45R, W47G, N74R, N77K, L79A, L93T, K98V, and M124Q mutations compared with a framework region of the VH set forth in SEQ ID NO: 48.

In a preferred embodiment, the nanobody or the antigen-binding fragment thereof at least comprises G26V, F27L, T28N, F29L, V37F, G44E, L45R, W47G, N74R, N77K, L79A, R87K, A88P, and K98V mutations compared with a framework region of the VH set forth in SEQ ID NO: 48.

In some embodiments, the nanobody or the antigen-binding fragment thereof comprises a framework region sequence at least having a mutation, compared with a framework region of the VH set forth in SEQ ID NO: 49, selected from the group consisting of: numbered in the natural order, mutations at positions E1, V2, A24, N35, V37, G44, L45, W47, K76, and L79. Preferably, in some embodiments, the nanobody or the antigen-binding fragment thereof comprises a framework region sequence at least having a mutation, compared with a framework region of the VH set forth in SEQ ID NO: 49, selected from the group consisting of: numbered in the natural order, E1Q, V2L, A24S, N35G, V37F, G44E, L45R, W47F, K76G, and L79V.

In a preferred embodiment, the nanobody or the antigen-binding fragment thereof at least comprises N35G, V37F, G44E, L45R, and W47F mutations compared with a framework region of the VH set forth in SEQ ID NO: 49.

In a preferred embodiment, the nanobody or the antigen-binding fragment thereof at least comprises A24S, N35G, V37F, G44E, L45R, and W47F mutations compared with a framework region of the VH set forth in SEQ ID NO: 49.

In a preferred embodiment, the nanobody or the antigen-binding fragment thereof at least comprises V2L, A24S, N35G, V37F, G44E, L45R, W47F, and E1Q mutations compared with a framework region of the VH set forth in SEQ ID NO: 49.

In a preferred embodiment, the nanobody or the antigen-binding fragment thereof at least comprises A24S, N35G, V37F, G44E, L45R, W47F, and K76G mutations compared with a framework region of the VH set forth in SEQ ID NO: 49.

In a preferred embodiment, the nanobody or the antigen-binding fragment thereof at least comprises A24S, N35G, V37F, G44E, L45R, W47F, and L79V mutations compared with a framework region of the VH set forth in SEQ ID NO: 49.

In some embodiments, the nanobody or the antigen-binding fragment thereof specifically binds to both human and monkey GUCY2C proteins; preferably, the nanobody or the antigen-binding fragment thereof binds to human and monkey GUCY2C proteins with a KD superior to 6.00E-7 M.

In some embodiments, the nanobody or the antigen-binding fragment thereof is: (1) a chimeric antibody or a fragment thereof; (2) a humanized antibody or a fragment thereof; or (3) a fully human antibody or a fragment thereof.

In some embodiments, the nanobody or the antigen-binding fragment thereof comprises or does not comprise an antibody heavy chain constant region. The antibody heavy chain constant region may be selected from human, Vicugna pacos, mouse, rat, rabbit, or sheep. The antibody heavy chain constant region may be selected from IgG, IgM, IgA, IgE, or IgD. The IgG may be selected from IgG1, IgG2, IgG3, or IgG4. The heavy chain constant region may be selected from an Fc region, a CH3 region, or an intact heavy chain constant region; preferably, the heavy chain constant region is a human Fc region; preferably, the nanobody or the antigen-binding fragment thereof is a heavy chain antibody.

In some embodiments, the nanobody or the antigen-binding fragment thereof is further conjugated to a therapeutic agent or a tracer. In some embodiments, the therapeutic agent is selected from a drug, a toxin, a radioisotope, a chemotherapeutic agent, or an immunomodulator. In some embodiments, the tracer is selected from a radiocontrast medium, a paramagnetic ion, a metal, a fluorescent label, a chemiluminescent label, an ultrasound contrast agent, and a photosensitizer.

In another aspect, the present application provides a multispecific molecule, wherein the multispecific molecule comprises any one of the foregoing nanobodies or the antigen-binding fragment thereof. Preferably, the multispecific molecule further comprises a nanobody or an antigen-binding fragment thereof specifically binding to an antigen other than GUCY2C or binding to a GUCY2C epitope different from that of any one of the foregoing nanobodies or the antigen-binding fragment thereof.

In some embodiments, the antigen other than GUCY2C may be an antigen on the surface of a T cell, a B cell, a natural killer cell, a dendritic cell, a macrophage, a monocyte, or a neutrophil. In a preferred embodiment, the antigen other than GUCY2C may be selected from: CD96, PD-1, PD-L1, PD-L2, OX40, OX40L, LAG-3, TIM3, VISTA, CD3, CD3γ, CD3δ, CD3ε, CD3ζ, CD27, CD28, CD28H, CD16, CD16A, CD32B, VEGF, NKG2D, NKp30, NKp46, NKp44, CD19, CD20, CD40, CD47, 4-1BB, ICOS, OX40, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WTi, NY-ESO-1, MAGE3, ASGPRI, TGFβ-trap, IL-2, IL-15, IL-21, IL-18, or CDH16.

In a preferred embodiment, the multispecific molecule may be bispecific, trispecific, or tetraspecific, and more preferably, the multispecific molecule may be divalent, tetravalent, or hexavalent.

In some embodiments, the multispecific molecule is a tandem scFv, a bifunctional antibody (Db), a single chain bifunctional antibody (scDb), a dual affinity retargeting (DART) antibody, a F(ab′)₂, a dual variable domain (DVD) antibody, a knobs-into-holes (KiH) antibody, a dock-and-lock (DNL) antibody, a chemically cross-linked antibody, a heteropolymeric nanobody, or a heteroconjugate antibody.

In another aspect, the present application provides a chimeric antigen receptor (CAR), wherein the chimeric antigen receptor at least comprises an extracellular antigen-binding domain, a transmembrane domain, and an intracellular signaling domain; the extracellular antigen-binding domain comprises any one of the foregoing nanobodies or the antigen-binding fragment thereof.

In another aspect, the present application provides an immune effector cell, wherein the immune effector cell expresses the chimeric antigen receptor described above or comprises a nucleic acid fragment encoding the chimeric antigen receptor; preferably, the immune effector cell is selected from a T cell, a natural killer cell (NK cell), a natural killer T cell (NKT cell), a double negative T cell (DNT cell), a monocyte, a macrophage, a dendritic cell, or a mast cell, and the T cell is preferably selected from a cytotoxic T cell, a regulatory T cell, or a helper T cell; preferably, the immune effector cell is an auto-immune effector cell or an allogeneic immune effector cell.

In another aspect, the present application provides an isolated nucleic acid fragment, wherein the nucleic acid fragment encodes any one of the foregoing nanobodies or the antigen-binding fragment thereof, or the multispecific molecule, or the chimeric antigen receptor.

In another aspect, the present application provides a vector, wherein the vector comprises the nucleic acid fragment.

In another aspect, the present application provides a host cell, wherein the host cell comprises the vector; preferably, the cell is a prokaryotic cell or a eukaryotic cell, such as a bacterium (e.g., E. coli), a fungus (e.g., yeast), an insect cell, or a mammalian cell (e.g., a CHO cell line or a 293T cell line).

In another aspect, the present application provides a method for preparing any one of the foregoing nanobodies or the antigen-binding fragment thereof, or multispecific molecules, wherein the method comprises culturing the host cell, and isolating a nanobody or an antigen-binding fragment thereof expressed by the cell, or isolating a multispecific molecule expressed by the cell.

In another aspect, the present application provides a method for preparing the immune effector cell, wherein the method comprises introducing a nucleic acid fragment encoding the CAR into the immune effector cell; optionally, the method further comprises initiating expression of the CAR in the immune effector cell.

In another aspect, the present application provides a pharmaceutical composition, wherein the pharmaceutical composition comprises any one of the foregoing nanobodies or the antigen-binding fragment thereof, multispecific antibodies, immune effector cells, nucleic acid fragments, vectors, or host cells, or a product prepared by any one of the foregoing methods; optionally, the pharmaceutical composition further comprises a pharmaceutically acceptable carrier, diluent, or adjuvant.

The pharmaceutically acceptable carrier is a carrier that does not decrease the viability and the function of immune cells, and does not affect the specific binding of an antibody or an antigen-binding fragment thereof to an antigen, including but not limited to cell culture media, buffers, physiological saline, balanced salt solutions, and the like. Examples of buffers include isotonic phosphates, acetates, citrates, borates, carbonates, and the like. In a specific embodiment, the pharmaceutically acceptable carrier is a phosphate-buffered saline containing 1% serum.

In another aspect, further provided is use of any one of the foregoing nanobodies or the antigen-binding fragment thereof, multispecific molecules, immune effector cells, nucleic acid fragments, vectors, host cells, products prepared by any one of the foregoing methods, or pharmaceutical compositions disclosed herein in preparing a medicament for preventing and/or treating a tumor, wherein the tumor may be selected from colorectal cancer, gastric cancer, small intestine cancer, esophageal cancer, pancreatic cancer, lung cancer, soft tissue sarcoma, and neuroendocrine tumor.

In another aspect, the present application provides a method for preventing and/or treating a tumor, wherein the method comprises administering to a patient in need thereof an effective amount of any one of the foregoing nanobodies or the antigen-binding fragment thereof, multispecific molecules, immune effector cells, nucleic acid fragments, vectors, host cells, products prepared by any one of the foregoing methods, or pharmaceutical compositions, wherein the tumor is selected from colorectal cancer, gastric cancer, small intestine cancer, esophageal cancer, pancreatic cancer, lung cancer, soft tissue sarcoma, and neuroendocrine tumor.

In another aspect, the present application further provides any one of the foregoing nanobodies or the antigen-binding fragment thereof, multispecific molecules, immune effector cells, nucleic acid fragments, vectors, host cells, products prepared by any one of the foregoing methods, or pharmaceutical compositions for use in preventing and/or treating a tumor, wherein the tumor is selected from colorectal cancer, gastric cancer, small intestine cancer, esophageal cancer, pancreatic cancer, lung cancer, soft tissue sarcoma, and neuroendocrine tumor.

In another aspect, the present application provides a kit, wherein the kit comprises any one of the foregoing nanobodies or the antigen-binding fragment thereof, multispecific antibodies, immune effector cells, nucleic acid fragments, vectors, host cells, products prepared by any one of the foregoing methods, or pharmaceutical compositions.

In another aspect, the present application provides a method for detecting GUCY2C expression, comprising contacting a sample to be tested with any one of the foregoing nanobodies or the antigen-binding fragment thereof in a condition allowing formation of a complex by any one of the foregoing nanobodies or the antigen-binding fragment thereof and GUCY2C.

In another aspect, the present application provides a method for inhibiting the proliferation or migration of a cell expressing GUCY2C in vitro, comprising contacting the cell with any one of the foregoing nanobodies or the antigen-binding fragment thereof in a condition allowing formation of a complex by any one of the foregoing nanobodies or the antigen-binding fragment thereof and GUCY2C.

The present application will be further described with reference to specific examples, and the advantages and features of the present application will become more apparent with the description.

Experimental procedures without specified conditions in the examples are conducted according to conventional conditions or conditions recommended by the manufacturers. Reagents or instruments without specified manufacturers used herein are conventional products that are commercially available.

The examples of the present application are exemplary only, and do not limit the scope of the present application in any way. It will be understood by those skilled in the art that various modifications or substitutions may be made to the technical solutions of the present application in form and details without departing from the spirit and scope of the present application, and that these modifications and substitutions shall fall within the protection scope of the present application.

### EXAMPLE

### Example 1. Preparation of Recombinant Protein and Control Antibody and Purification Method Thereof

### 1.1 Design and Expression of Recombinant Protein

GUCY2C recombinant proteins were constructed according to human, cynomolgus monkey, and mouse amino acid sequences: Human GUCY2C protein (UniProt No: P25092) was used as a template sequence to design a tagged fusion protein, which was separately cloned into a pTT5 vector (Youbio, VT2202). A GUCY2C plasmid was constructed and transiently expressed in Expi 293F cells (Gibco, A14527) to give the antigen and the protein for detection in this example. The method for preparing the cynomolgus monkey and mouse proteins was the same as the method for preparing the human recombinant protein. The cynomolgus monkey GUCY2C sequence was from Uniprot No: AOA2K5TZ15. The mouse GUCY2C sequence was from Uniprot: Q3UWA6. The specific sequence information of the recombinant protein is shown below: Human GUCY2C ECD (his-tagged human GUCY2C protein extracellular domain fusion protein) (SEQ ID NO: 1):

SQVSQNCHNGSYEISVLMMGNSAFAEPLKNLEDAVNEGLEIVRGRLQNA
GLNVTVNATFMYSDGLIHNSGDCRSSTCEGLDLLRKISNAQRMGCVLIG
PSCTYSTFQMYLDTELSYPMISAGSFGLSCDYKETLTRLMSPARKLMYF
LVNFWKTNDLPFKTYSWSTSYVYKNGTETEDCFWYLNALEASVSYFSHE
LGFKVVLRQDKEFQDILMDHNRKSNVIIMCGGPEFLYKLKGDRAVAEDI
VIILVDLFNDQYFEDNVTAPDYMKNVLVLTLSPGNSLLNSSFSRNLSPT
KRDFALAYLNGILLFGHMLKIFLENGENITTPKFAHAFRNLTFEGYDGP
VTLDDWGDVDSTMVLLYTSVDTKKYKVLLTYDTHVNKTYPVDMSPTFTW
KNSKLPNDITGRGPQHHHHHH*

Cyno GUCY2C ECD (his-tagged cynomolgus monkey GUCY2C protein extracellular domain fusion protein) (SEQ ID NO: 2):

SQVSQNCHNGSYEISVLMMDNSAFAEPLENVEDAVNEGLEIVRGRLQNA
GLNVTVNASFMYSDGLIHNSGDCRSSTCEGLDLLRKISNAKRMGCVLMG
PSCTYSTFQMYLDTELSYPMISAGSFGLSCDYKETLTRLMSPARKLTYF
LVNFWKTNDLPFKTYSWSTSYVYKNGTESEDCFWYLNALEASVSYFSHE
LSFKLVLRQDKEFQDILMDHNRKSNVIVMCGDPEFLYKLKGDRAVAEDI
VIILVDLFNDQYFEDNVTAPDYMKNVLVLTQSPGNSLLNSSFSRNLSPT
KRDFALAYLNGILLFGHMLKTFLENGENITTPKFAHAFRNLTFEGYDGP
VTLDDWGDVDSTMVLLYTSVDTKKYKVLLTYDTHVNQTNPVDMSPTFTW
KNSKLPNDITDRGPQHHHHHH*

Mouse GUCY2C ECD (his-tagged mouse GUCY2C protein extracellular domain fusion protein) (SEQ ID NO: 3):

VFWASQVRQNCRNGSYEISVLMMDNSAYKEPMQNLREAVEEGLDIVRKR
LREADLNVTVNATFIYSDGLIHKSGDCRSSTCEGLDLLREITRDHKMGC
ALMGPSCTYSTFQMYLDTELNYPMISAGSYGLSCDYKETLTRILPPARK
LMYFLVDFWKVNNASFKPFSWNSSYVYKNGSEPEDCFWYLNALEAGVSY
FSEVLNFKDVLRRSEQFQEILTGHNRKSNVIVMCGTPESFYDVKGDLQV
AEDTVVILVDLFSNHYFEENTTAPEYMDNVLVLTLPSEQSTSNTSVAER
FSSGRSDFSLAYLEGTLLFGHMLQTFLENGENVTGPKFARAFRNLTFQG
FAGPVTLDDSGDIDNIMSLLYVSLDTRKYKVLMKYDTHKNKTIPVAENP
NFIWKNHKLPNDVPGLGPQILMHHHHHH*

### 1.2 Design, Expression, and Purification of Control Antibody

The control antibodies used in this example are all derived from published patent sequences. The PF1608 antibody was derived from published patent application No. WO2019224716A2, and the 5F9 antibody was derived from published patent application No. WO2017136693A1. Unless otherwise indicated, the PF1608 and 5F9 control antibodies were for recombinant expression using the human IgG1+x subtype. The nanobodies and humanized antibodies thereof described herein were all for recombinant expression using the human Fc fusion form.

The expression and purification process of the control antibody was as follows: the antibody sequence gene was synthesized and cloned into an expression vector pTT5. Expi293F cells (purchased from Gibco, A14527) were transiently transfected, and the cell supernatant was collected after the cells were cultured on a shaker at 37° C. for 7 days for protein A antibody purification. See “1.3.2 Purification of control antibody by Protein A affinity chromatography” for the purification process. The resulting control antibodies were designated as PF1608-hIgG1 and 5F9-hIgG1. The specific sequence information of the antibodies is shown in Table 1.

TABLE 1
Sequence listing of control antibodies

| Antibody name | Sequence No. | Sequence |
|---|---|---|
| 5F9-hIgG1 | SEQ ID NO. 4 | QVQLQQWGAGLLKPSETLSLTCAVFGGSFSGYYWSWIRQPPGKGLEWIG |
| | | EINHRGNTNDNPSLKSRVTISVDTSKNQFALKLSSVTAADTAVYYCARER |
| | | GYTYGNFDHWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK** |
| 5F9-hIgK | SEQ ID NO. 5 | EIVMTQSPATLSVSPGERATLSCRASQSVSRNLAWYQQKPGQAPRLLIYG |
| | | ASTRATGIPARFSGSGSGTEFTLTIGSLQSEDFAVYYCQQYKTWPRTFGQG |
| | | TNVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC** |
| PF1608-hIgG1 | SEQ ID NO. 6 | EVQLVESGGGLVQPGGSLRLSCAASGFTFSSYWMHWVRQAPGKGLEWI |
| | | GEIKPSNELTNVHEKFKDRFTISVDKAKNSAYLQMNSLRAEDTAVYYCTR |
| | | TITTTEGYWFFDVWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGK** |
| PF1608-hIgK | SEQ ID NO. 7 | DIQLTQSPSSLSASVGDRVTITCRASESVDYYGSSLLQWYQQKPGKAPKL |
| | | LIYAASKLASGVPSRFSGSGSGTDFTLTISSLQPEDFATYYCQQTRKAYTFG |
| | | QGTKLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC** |
| Human Fc | SEQ ID NO. 8 | EPKSSDKTHTCPPCPAPELLGGPSVFLFPPKPKDTLMISRTPEVTCVVVDV |
| (C220S) | | SHEDPEVKFNWYVDGVEVHNAKTKPREEQYNSTYRVVSVLTVLHQDWL |
| sequence | | NGKEYKCKVSNKALPAPIEKTISKAKGQPREPQVYTLPPSREEMTKNQVS |
| | | LTCLVKGFYPSDIAVEWESNGQPENNYKTTPPVLDSDGSFFLYSKLTVDKS |
| | | RWQQGNVFSCSVMHEALHNHYTQKSLSLSPGK** |

### 1.3 Purification of Recombinant Protein and Purification of Control Antibody

1.3.1 Purification of recombinant protein on nickel column After the relevant recombinant protein was constructed and expressed according to the step “1.1 Design and expression of recombinant protein”, purification was performed as follows: the cell expression supermatant sample was centrifuged at high speed to remove impurities, and a nickel column was equilibrated with 20 mM PBS+500 mM NaCl solution and washed with 2-5 column volumes. The culture supermatant was loaded onto a Ni affinity chromatographic column (purchased from GE Healthcare), and meanwhile, the changes in UV absorption value (A280 nm) were monitored using an ultraviolet (UV) detector. The column was washed with an equilibration buffer until the A280 reading dropped to the baseline, then gradient elution was separately performed using equilibration buffers containing 10 mM, 20 mM, 40 mM, 90 mM, 250 mM, and 500 mM imidazole, and each of the elution peaks was collected. The component of the target protein was determined according to the SDS-PAGE gel image. The eluted product comprising the target protein was collected and concentrated, which may be further purified by gel chromatography Superdex200 (GE) with PBS as the mobile phase to remove aggregates and impurity protein peaks, and the elution peak of the target product was collected. The obtained proteins were identified by electrophoresis, peptide mapping, and LC-MS, and then aliquoted for later use after they were determined to be correct. The proteins obtained by purification by this protocol include human GUCY2C-His, monkey GUCY2C-His, and murine GUCY2C-His.

### 1.3.2 Purification of Control Antibody by Protein a Affinity Chromatography

The obtained control antibody sequences were cloned into eukaryotic expression vectors pTT5 (Youbio, VT2202, Fc (C220S) sequence) with a human Fc tag, respectively. Expi293F cells were transiently transfected by PEI (Polysciences, 24765-1), cultured for 7 days, and then centrifuged at high speed to collect the cell culture supernatant expressing the antibodies. The Protein A (Bestchrom, AA0273) protein chromatographic column was washed with 3-5 column volumes of 0.1 M NaOH, and then washed with 3-5 column volumes of pure water. The chromatographic column was equilibrated using 3-5 column volumes of 1× PBS (pH 7.4) buffer system as the equilibration buffer. The cell supernatant was loaded on the column at a low flow rate for binding, with the flow rate controlled to allow for a retention time of about 1 min or longer. After the binding was completed, the chromatographic column was washed with 3-5 column volumes of 1× PBS (pH 7.4) until the UV absorbance fell back to baseline. The sample was eluted with a 50 mM citric acid/sodium citrate (pH 3.0-3.5) buffer, the elution peaks were collected by UV detection, and the eluted product was rapidly adjusted to pH 5-6 with 1 M Tris-HCl (pH 8.0) and temporarily stored.

For the eluted product, solution exchange may be performed by methods well known to those skilled in the art, such as ultrafiltration concentration using an ultrafiltration tube and exchange of the solution to a desired buffer system, or exchange with a desired buffer system by molecular exclusion (e.g., G-25 desalination), or removal of polymer components from the eluted product using a high-resolution molecular exclusion column such as Superdex 200 to improve sample purity. After protein A protein affinity purification, an antibody with a human Fc tag eluted from the chromatographic column was collected to give a corresponding purified antibody.

### Example 2. Construction of Stably Transfected Cell Strain

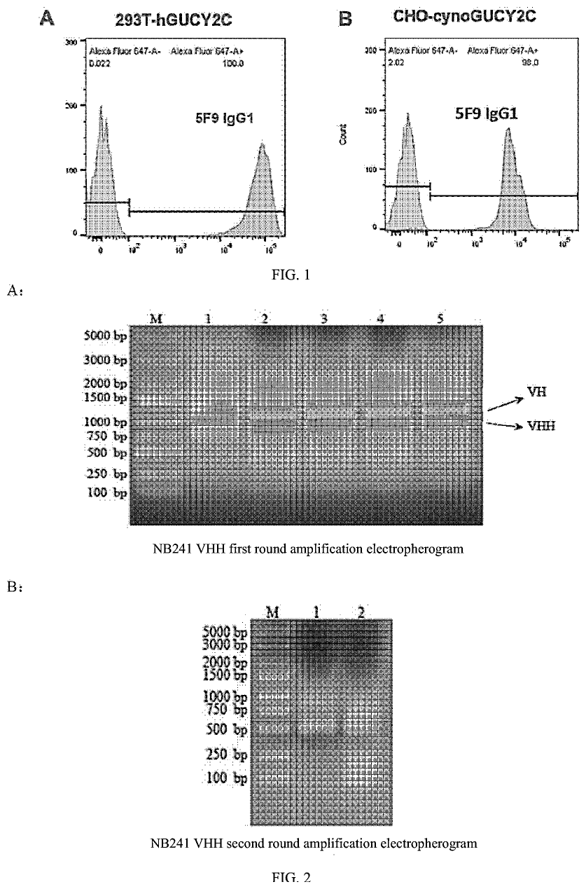

A nucleotide sequence encoding a full-length amino acid sequence of human GUCY2C protein (UniProt: P25092) was cloned into a pcDNA3.1 vector (purchased from Clontech), and a plasmid was prepared. After the plasmid transfection (Lipofectamine® 3000 Transfection Kit, purchased from Invitrogen, Cat. No. L3000-015) of an 293T cell, selective culture was performed in DMEM medium containing 10 μg/mL puromycin for 2 weeks, and positive monoclonal cells were sorted into 96 well plates on a flow cytometer FACS AriaII (purchased from BD Biosciences) using the human GUCY2C antibody (5F9, self-produced) and an anti-human IgG (H+L) antibody (Jackson, Cat. No. 109-605-088) and cultured under the conditions of 37° C. and 5% (v/v) C02. After about 2 weeks, some of the monoclonal wells were selected for amplification. The amplified clones were screened by flow cytometry. Monoclonal cell lines with better growth and higher fluorescence intensity were selected for further expansion and cryopreserved in liquid nitrogen. The resulting cell strain was designated as 293T-hGUCY2C. The expression measured by FACS is shown as A in FIG. 1.

A nucleotide sequence encoding a full-length amino acid sequence of cynomolgus monkey GUCY2C protein (UniProt: A0A2K5TZ15) was cloned into a pcDNA5-FRT vector (purchased from General), and a plasmid was prepared. After the plasmid transfection of the FlpinCHO cell line (purchased from Invitrogen), selective culture was performed in F12 medium containing 800 μg/mL hygromycin for 2 weeks. The resulting cell strain was designated as CHO-cyno GUCY2C, which was assayed by FACS using the GUCY2C positive control 5F9 antibody (shown as B in FIG. 1).

### Example 3. Preparation of Nanobody Against Guanylate Cyclase C Protein

### 3.1 Vicugna Pacos Immunization and Serum Titer Assay

1 Vicugna pacos of 1.5-3 years old was selected, and 10 mL of blood was collected before immunization, which was left as the negative control serum. According to the primary immunization dose doubling principle, 0.5 mg of the human guanylate cyclase C protein was mixed well with Freund's complete adjuvant (purchased from Sigma, F5881) and then injected subcutaneously in multiple spots into the neck for immunization. After two weeks, a second immunization was performed. 0.25 mg of the protein was mixed with Freund's incomplete adjuvant and then injected subcutaneously in multiple spots into the neck for immunization. After one week, the serum was taken for titer assay. In a third immunization, 0.25 mg of the protein was mixed with Freund's incomplete adjuvant and then injected subcutaneously in multiple spots into the neck for immunization. After one week, the serum was taken for titer assay. In a fourth immunization, 0.25 mg of the protein was mixed with Freund's incomplete adjuvant and then injected subcutaneously in multiple spots into the neck for immunization. After one week, the serum was taken for titer assay. The titer and specificity of the antibody in the serum against human, murine, and monkey guanylate cyclase C proteins were assayed using enzyme-linked immunosorbent assay (ELISA). The results of the Vicugna pacos serum titer of the third and fourth immunizations are shown in Table 2, in which the data are OD450 nm values.

TABLE 2
Serum antibody titer of Vicugna pacos after being immunized with a guanylate cyclase
C protein assayed by ELISA

| | OD450 | | | | | |
|---|---|---|---|---|---|---|
| | Batch | | | | | |
| | Third | Fourth | Third | Fourth | Third | Fourth |
| Dilution | immunization | immunization | immunization | immunization | immunization | immunization |
| ratio | Human | Human | Mouse | Mouse | Cyno | Cyno |
| 1:8K | 3.73 | 3.57 | 1.64 | 1.54 | 3.87 | 4.02 |
| 1:16K | 3.17 | 3.24 | 1.13 | 1.32 | 3.21 | 3.56 |
| 1:32K | 2.58 | 2.22 | 0.86 | 0.75 | 2.59 | 2.72 |
| 1:64K | 1.65 | 1.42 | 0.43 | 0.36 | 1.44 | 1.60 |
| 1:128K | 0.79 | 0.78 | 0.22 | 0.21 | 0.85 | 0.85 |
| 1:256K | 0.59 | 0.53 | 0.15 | 0.14 | 0.51 | 0.53 |
| 1:528K | 0.30 | 0.31 | 0.09 | 0.09 | 0.27 | 0.30 |
| BLANK | 0.05 | 0.04 | 0.05 | 0.05 | 0.05 | 0.05 |

### 3.2 Construction of Nanobody Library

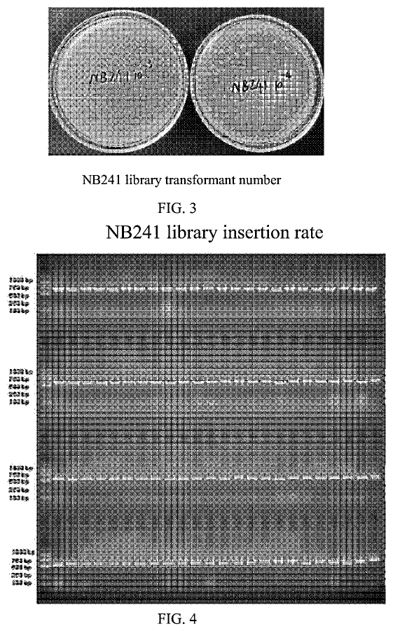

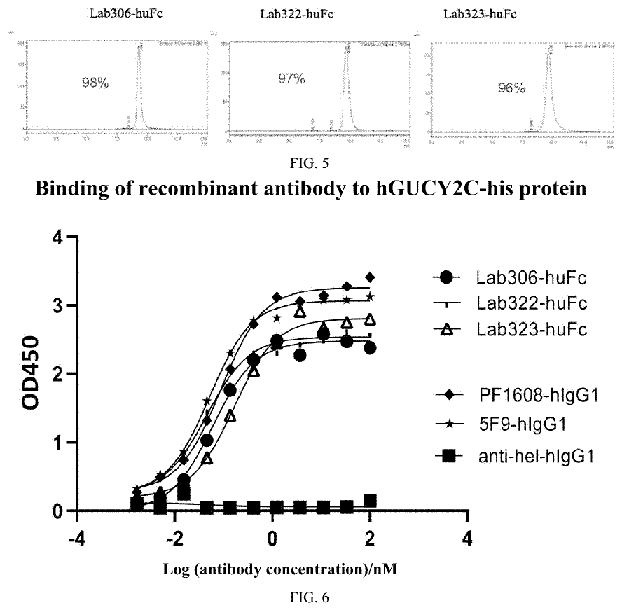

A total of 50 mL peripheral blood was collected from Vicugna pacos after the third and fourth immunizations, and PBMCs were isolated using a lymphocyte isolation medium. The total RNA was extracted after three and four immunizations using RNAiso Plus reagent (purchased from Takara, 9108/9109). A total of 5 pg RNA was transcribed according to the instructions of the reverse transcription kit PrimeScript™ II 1t Strand cDNA Synthesis Kit (Takara, 6210A). The nanobody (VHH) fragments were amplified by nested PCR using cDNA as the template. FIG. 2 shows the results of the first and second rounds of amplification of the VHH fragment. The results show that the size of the target band after the first round of amplification is about 750 bp, and the size of the target band after the second round of amplification is about 500 bp. After the PCR product was purified, it was ligated into the phage display vector pComb3Xss (Chengdu NBbiolab, Co. Ltd., P001) using a restriction endonuclease SfiI (purchased from NEB, R0123L). The ligation product was then electrotransformed into TG1 competent cells for 10 times, and 1 mL of 2YT medium (purchased from Sangon, A507019-0250) was immediately added to the cuvette after electroporation to thaw the cells. The electroporation product was pipetted, and the cuvette was washed with 2YT medium (purchased from Sangon, A507019-0250) to give 100 mL of a TG1 cell thawing product in total. The TG1 cell thawing product was thawed at 37° C. at 180 rpm for 45 min. 100 mL of the TG1 bacterium solution was serially diluted by 10³ times and 10⁴ times. The number of transformants in the nanobody library was determined. The TG1 bacterium solutions diluted by 10³ times and 10⁴ times were coated on a 90 mm plate, and the remaining bacterium solution was centrifuged and resuspended with 8 mL of 2YT medium (purchased from Sangon, A507019-0250). The bacterium solution was coated on 8 200 mm plates. The results of the number of transformants on the gradient dilution are shown in FIG. 3. A total of 158 clones were on the plate “NB241 10⁻⁴” for determining the number of transformants in the nanobody library. The size of the reservoir was calculated to be 1.9×10⁹. FIG. 4 shows that 96 clones were randomly picked from the titer plate for the number of transformants in the library for identification. The band size of the target protein was about 500 bp. The results show that 96 clones are all positive, indicating that the insertion rate is 100%.

The forward primer of the first round of amplification LD-F:

(SEQ ID NO. 9)
CTTGGTGGTCCTGGCTGC

The reverse primer of the first round of amplification CH2-R:

(SEQ ID NO. 10)
GGTACGTGCTGTTGAACTGTTCC

The forward primer of the second round of amplification was Primer F:

(SEQ ID NO. 11)
CATGCCATGACTGTGGCCCAGGCGGCCCAGKTGCAGCTCGTGGAGTC

The reverse primer of the second round of amplification Primer R1:

(SEQ ID NO.12)
CATGCCATGACTCGCGGCCGGCCTGGCCATGGGGGTCTTCGCTGTGGTG
CG

The reverse primer of the second round of amplification Primer R2:

(SEQ ID NO. 13)
CATGCCATGACTCGCGGCCGGCCTGGCCGTCTTGTGGTTTTGGTGTCT
TGGG

### 3.3 Panning of Nanobodies Against GUCY2C Protein

For the first round of biopanning, three tubes of A, B, and C were prepared, 100 μL of streptavidin-conjugated Dynabeads (purchased from Invitrogen) and the Vicugna pacos phage antibody library NB241 described above were first added to tube A, and 100 μL of streptavidin-conjugated Dynabeads were first added to tube B. Then a blocking buffer, i.e., PBS phosphate-buffered saline containing 20% (w/v) skim milk powder, was separately added to the three tubes for blocking at room temperature for 2 h. The liquid in tube C was discarded, the supernatant collected after centrifugation of tube A was added, then 4 μg of biotinylated human GUCY2C-His protein was added, and biotinylation was performed according to kit instructions (purchased from Dojindo, LK03). The mixture was incubated at room temperature for 2 h while shaking. Moreover, a control tube was set, only non-biotinylated human GUCY2C-His protein was added, and the mixture was incubated at room temperature for 1 h while shaking. Tube B was centrifuged to give the blocked magnetic beads, the incubated mixed solution was added, and the mixture was incubated at room temperature for 15 min while shaking. The tube was placed on the magnetic rack for 30 s, washed 10 times with 1 mL of PBST, i.e., blocking buffer containing 0.01% (v/v) Tween-20, and washed 1 time with PBS buffer. After washing, 500 μL of 10 μg/mL pancreatin was added to each tube, and the mixture was incubated at 37° C. for 15 min to elute phages binding to the biotinylated human GUCY2C-His protein. 250 μL of a pancreatin solution was added to 4 mL of E. coli TGT (purchased from LUCIGEN) in the logarithmic growth phase, and the mixture was incubated at 37° C. for 30 min to give a TGT culture solution. The TGT culture solution was serially diluted, coated on a plate, and cultured at 37° C. overnight. The number of the resulting clones binding to the biotinylated human GUCY2C-His protein and clones of the control tube was calculated, and 48 clones were selected for sequencing. Meanwhile, the clones on the plate were washed with 2YT medium (purchased from Sangon, 2YT medium was prepared by adding 31 g of 2YT medium powder to 1 L of water, adjusting the pH to 7.0 with NaOH, and autoclaving), collected, inoculated into a fresh medium, and cultured at 37° C. to the logarithmic phase. Helper phages M13KO7 (purchased from NEB, Cat. No. N0315S) were added with a ratio of helper phage to E. coli TGT of 20:1. The mixture was mixed well and left to stand at 37° C. for 30 min. Then the mixture was cultured at 37° C. for 30 min while shaking and centrifuged at 4000 rpm for 10 min. The cells were collected, a fresh 2YT medium containing ampicillin and kanamycin resistance was added, and the mixture was cultured at 30° C. overnight while shaking. The mixture was centrifuged at 5000 rpm for 20 min, the supernatant was collected, ¼ of the supernatant volume of a 2.5 M NaCl solution containing 20% PEG6000 was added, and the mixture was left on ice overnight. The mixture was centrifuged at 5000 rpm at 4° C. for 30 min, and the phage pellet was collected and dissolved in PBS buffer. The mixture was centrifuged at 10000 rpm for 10 min to remove the residual cell debris, and the supernatant was collected for the next round of biopanning.

The steps of the second and third rounds of biopanning were consistent with those of the first round. In the second round, VHH antibody sequences that specifically bind to the biotinylated monkey GUCY2C-His protein were enriched. In the third round, VHH antibody sequences that specifically bind to the biotinylated human GUCY2C-His protein were enriched. After multiple rounds of panning, the positive phages were continuously enriched in the panning process, so that a nanobody with good specificity and high affinity was screened out.

### 3.4 Screening of Positive Clones by Enzyme-Linked Immunosorbent Assay

From the second and third rounds of plates, single clones were selected and cultured in a 96-well plate. 200 μL of 2YT medium containing antibiotics and 1% glucose was added to each well, and the mixture was cultured at 250 rpm at 37° C. overnight while shaking. 10 μL of the overnight culture was added to 100 μL of 2YT medium containing antibiotics and 0.5% glucose, and the mixture was cultured until the OD600 was 0.4-0.6. Helper phages were added at an infection ratio of 20:1, and the mixture was left to stand at 37° C. for 30 min. Then the mixture was cultured at 37° C. for 30 min while shaking, 400 μL of 2YT medium containing antibiotics was added, and the mixture was cultured at 30° C. overnight. The next day, the mixture was centrifuged at 5000 rpm at 4° C. for 20 min, and the resulting supernatant was used for monoclonal ELISA identification.

The human, murine, and monkey GUCY2C proteins were diluted with carbonate buffer at pH 9.6 to a final concentration of 2 μg/mL, added to enzyme-labeled wells at 50 μL/well, and coated at 4° C. overnight. Then 50 μL of the phage culture bacterium solution supernatant and a horseradish peroxidase-labeled anti-M13 antibody (purchased from Sino Biological, 11973-MM05T-H) diluted at a ratio of 1:4000 were added to each well. A TMB color-developing solution (purchased from KPL, 52-00-02) was added for color developing after washing the plates, and the optical density was measured at 450 nm. Positive clones binding to both human and monkey GUCY2C proteins were selected for FACS assay.

### 3.5 Screening of Phage Clone Binding to Cell by Flow Cytometry Assay (FACS)

The obtained 293T-hGUCY2C stably transfected cell strain and the control cells 293T and the endogenous cells HT55 (purchased from Nanjing Cobioer, Cat. No. 2811) were expanded in a T-175 cell culture flask until the confluence was 90%. The medium was completely pipetted off. The cells were washed 1 time with PBS buffer, then treated with Trypsin-EDTA (purchased from Gibco, Cat. No. 25200072) and collected. After counting, the cells were washed 2 times with PBS phosphate-buffered saline, diluted to 2×10⁶ cells per mL, and added to a 96-well FACS reaction plate at 50 μL/well. 1% (w/w) fetal bovine serum was added to the PBS phosphate-buffered saline as an FACS buffer, and the plate was centrifuged at 1500 rpm at 4° C. and washed 2 times. 50 p L of the phage supernatant was added to each well, and the mixture was incubated on ice for 1 h. After the plate was centrifuged and washed 3 times with an FACS buffer, 50 μL of an anti-M13 antibody (purchased from Sino Biological, Cat. No. 11973-MM05T) diluted at a ratio of 1:1000 was added to each well, and the mixture was incubated on ice for 1 h. After the plate was centrifuged and washed 3 times with an FACS buffer, a fluorescently (Alexa 647)—labeled secondary antibody (purchased from Jackson Immuno, Cat. No. 115-605-003) was added to each well, and the mixture was incubated on ice for 1 h. The plate was centrifuged and washed 3 times with an FACS buffer. The cells were suspended in 100 μL of an FACS buffer and tested by FACS (purchased from BD, FACS Verse), and the results were analyzed.

Through multiple rounds of optimization and screening, 3 positive clones capable of recognizing human and monkey GUCY2C simultaneously were selected therefrom and designated as Lab306, Lab322, and Lab323, respectively. The CDRs of their sequences were analyzed using KABAT, Chothia, or IMGT software, respectively. The corresponding sequence information is shown in Tables 3-4 below, wherein Table 3 shows the antibody sequences represented by the amino acids of 3 Vicugna pacos nanobody molecules, and Table 4 shows the results of IMGT, Kabat, and Chothia analysis of the CDRs of 3 Vicugna pacos nanobody molecules.

TABLE 3
Amino acid specific sequence information of anti-GUCY2C Vicugna pacos nanobody

| Antibody | | |
|---|---|---|
| name | Sequence No. | Amino acid sequence |
| Lab306 | SEQ ID NO. 14 | QLQLVESGGGLVQPGGSLRLSCVTSGFTLDDYAIGWFRQAPGKERE |
| | | GVICLTSGGLPKYADSVKGRFTISRDNAKNTVYLRMDSLKPDDTAV |
| | | YYCAASGATSRWNCPPQYDHWGQGTQVTVSS |
| Lab322 | SEQ ID NO. 15 | QLQLVESGGGLVQPGGSLRLSCAASVLNLDYYAITWFRQAPGKERE |
| | | GVSCIQGRGTSTNYADSVKGRFTISRDRAKKTAYLQMNSLKPEDTA |
| | | TYYCAVRYVSYGRNTACGLLPESVDHWGQGTQVTVSS |
| Lab323 | SEQ ID NO. 16 | QLQLVESGGGLVQPGGSLRLSCVSSGRTSTIHRMGWFRQAPGKERD |
| | | FVASISRVSSTTYYADSVKGRFTISRDNAGNTVYLQMNSLKPEDTAV |
| | | YYCAASEYEVSPGKSKYWGQGTQVTVSS |

TABLE 4
Analysis of CDR specific sequence information of anti-GUCY2C Vicugna pacos
nanobody by IMGT, KABAT, and Chothia software
IMGT Analysis

| Antibody | | | | | Sequence | |
|---|---|---|---|---|---|---|
| No. | Sequence No. | HCDR1 | Sequence No. | HCDR2 | No. | HCDR3 |
| Lab306 | SEQ ID NO. 17 | GFTLDDYA | SEQ ID NO. 18 | LTSGGLP | SEQ ID | AASGATSRWNCPPQ |
| | | | | | NO. 19 | YDH |
| Lab322 | SEQ ID NO. 20 | VLNLDYYA | SEQ ID NO. 21 | IQGRGTST | SEQ ID | AVRYVSYGRNTACG |
| | | | | | NO. 22 | LLPESVDH |
| Lab323 | SEQ ID NO. 23 | GRTSTIHR | SEQ ID NO. 24 | ISRVSSTT | SEQ ID | AASEYEVSPGKSKY |
| | | | | | NO. 25 | |

Kabat analysis

| Antibody | | | | | Sequence | |
|---|---|---|---|---|---|---|
| No. | Sequence No. | HCDR1 | Sequence No. | HCDR2 | No. | HCDR3 |
| Lab306 | SEQ ID NO. 26 | DYAIG | SEQ ID NO. 27 | CLTSGGLPKY | SEQ ID | SGATSRWNCPPQYD |
| | | | | ADSVKG | NO. 28 | H |
| Lab322 | SEQ ID NO. 29 | YYAIT | SEQ ID NO. 30 | CIQGRGTSTN | SEQ ID | RYVSYGRNTACGLL |
| | | | | YADSVKQ | NO. 31 | PESVDH |
| Lab323 | SEQ ID NO. 32 | IHRMG | SEQ ID NO. 33 | SISRVSSTTYY | SEQ ID | SEYEVSPGKSKY |
| | | | | ADSVKG | NO. 34 | |

Chothia Analysis

| Antibody | | | | | Sequence | |
|---|---|---|---|---|---|---|
| No. | Sequence No. | HCDR1 | Sequence No. | HCDR2 | No. | HCDR3 |
| Lab306 | SEQ ID NO. 35 | GFTLDDY | SEQ ID NO. 36 | TSGGL | SEQ ID | SGATSRWNCPPQYD |
| | | | | | NO. 37 | H |
| Lab322 | SEQ ID NO. 38 | VLNLDYY | SEQ ID NO. 39 | QGRGTS | SEQ ID | RYVSYGRNTACGLL |
| | | | | | NO. 40 | PESVDH |
| Lab323 | SEQ ID NO. 41 | GRTSTIH | SBQ ID NO. 42 | SRVSST | SEQ ID | SEYEVSPGKSKY |
| | | | | | NO. 43 | |

### Example 4. Expression and Purification of Recombinant Nanobody and Assay on Affinity

### 4.1 Expression and Purification of Recombinant Nanobody

The obtained Vicugna pacos nanobody sequences were cloned into eukaryotic expression vectors pTT5 with an Fc tag, respectively. Expi293F cells (purchased from Gibco, A14527) were transiently transfected by PEI, cultured for 6 days, and then centrifuged at high speed to collect the cell culture supernatant expressing the antibodies. The antibodies were purified according to the purification method described in Example 1.3.2 to give corresponding recombinant nanobodies which were designated as Lab306-huFc, Lab322-huFc, and Lab323-huFc, respectively. The purified antibody was detected using an SEC-HPLC method. As shown in FIG. 5, the purities of the three antibodies were all greater than 95%.

### 4.2 Assay on Binding of Recombinant Antibodies to Human GUCY2C-his Protein by Enzyme-Linked Immunosorbent Assay (ELISA)

A human GUCY2C-his protein was diluted with PBS to a final concentration of 2 μg/mL and then added to a 96-well ELISA plate at 50 μL/well. The plate was sealed with a plastic film and incubated at 4° C. overnight. The next day, the plate was washed 2 times with PBST, and a blocking buffer [PBS+2% (w/w) BSA] was added for blocking at room temperature for 2 h. The blocking buffer was discarded, and the recombinant antibody and positive and negative control antibody with a starting concentration of 100 nM serially diluted by 3 folds were added at 50 μL/well. After incubation at 37° C. for 1 h, the plate was washed 3 times with PBST. A horseradish peroxidase (HRP)—labeled secondary antibody (purchased from Merck, Cat. No. AP113P) was added for incubation at 37° C. for 1 h, and the plate was washed 5 times with PBST. A TMB substrate was added at 50 μL/well for incubation at room temperature for 10 min, and then a stop solution (1.0 M HCl) was added at 50 μL/well. The OD450 nm values were read by an ELISA plate reader (Multimode Plate Reader, EnSight, purchased from Perkin Elmer). The binding activities of the recombinant antibodies Lab306-huFc, Lab322-huFc, and Lab323-huFc with human GUCY2C protein are shown in FIG. 6, indicating that all the purified antibodies bind effectively to human GUCY2C-his protein, wherein the negative control antibody hIgG1 is the antibody anti-he1-hIgG1 (purchased from Biointron, Cat. No. B117901) against hen egg lysozyme.

### 4.3 Assay on Binding of Recombinant Antibodies to Monkey GUCY2C-his and Murine GUCY2C-his Proteins by Enzyme-Linked Immunosorbent Assay (ELISA)

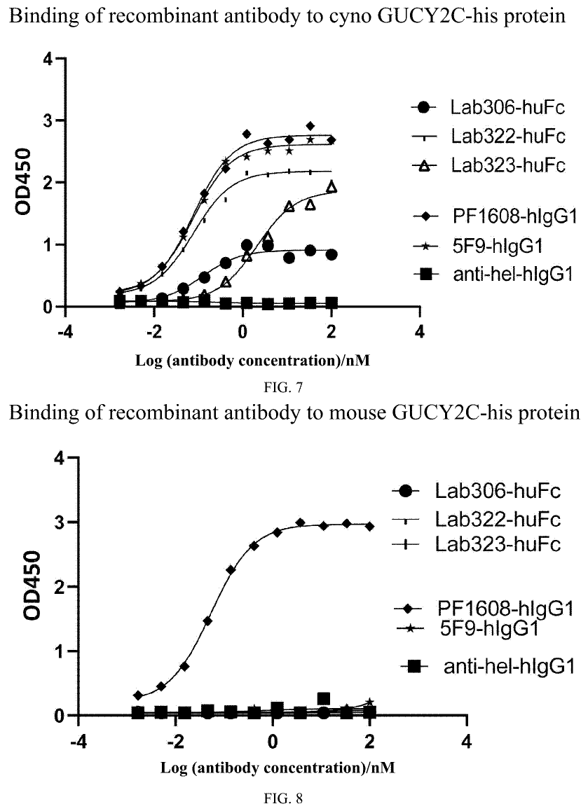

The monkey GUCY2C-his and murine GUCY2C-his proteins were subjected to ELISA and data analysis according to the method described in Example 4.2. The analysis results are shown in FIG. 7. The recombinant antibodies Lab306-huFc, Lab322-huFc, and Lab323-huFc have a relatively good binding activity with the monkey GUCY2C-his protein. The recombinant antibodies Lab306-huFc, Lab322-huFc, and Lab323-huFc have no binding activity with the murine GUCY2C-his protein (FIG. 8).

### 4.4 Assay on Binding of Recombinant Antibodies to Human GUCY2C by Flow Cytometry Assay (FACS)

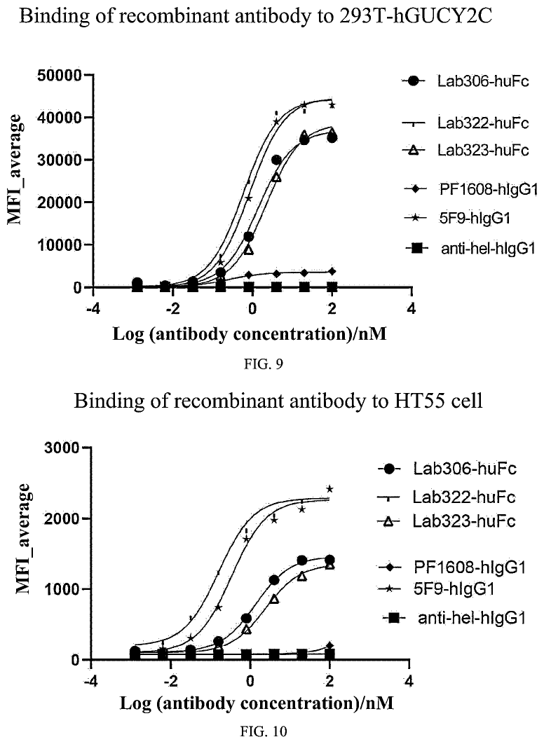

The stably transfected cell line 293T-hGUCY2C was expanded to the logarithmic growth phase in a T-175 culture flask, the medium was removed by pipetting, and the cells were washed 2 times with a PBS buffer and digested with trypsin. Then a complete medium was added to stop the digestion, and the cells were blown into a single cell suspension. After counting, the cells were centrifuged, and the cell pellet was resuspended to 2×10⁶ cells/mL in an FACS buffer (PBS+2% fetal bovine serum), and added to a 96-well FACS reaction plate at 100 μL/well. The plate was centrifuged, and the supernatant was discarded. An antibody sample to be tested (diluted in a 3-fold gradient from a starting concentration of 100 nM) was added at 50 μL/well and uniformly mixed with the cells, and the mixture was incubated at 4° C. for 1 h. After the plate was centrifuged and washed 3 times with a PBS buffer, 50 μL of Alexa Fluor® 647 AffiniPure Goat Anti-Human IgG, Fcγ fragment specific-labeled secondary antibodies (purchased from Jackson, Cat. No. 109-605-098) were added to each well, and the mixture was incubated at 4° C. for 1 h. The results were tested and analyzed by FACS (FACS Canto™, purchased from BD) after the plate was centrifuged and washed 3 times with a PBS buffer and resuspended with 100 μL of PBS. Data analysis was performed by software (FlowJo) to obtain the mean fluorescence intensity (MFI) of the cells. Then, analysis was performed by software (GraphPad Prism8), data were fitted, and EC50 values were calculated. As shown in FIG. 9, the recombinant antibodies Lab306-huFc, Lab322-huFc, and Lab323-huFc are all capable of binding specifically to 293T-hGUCY2C recombinant cells.

The binding of the recombinant antibody to the endogenous tumor cell HT55 was detected in the same manner. As shown in FIG. 10, the recombinant antibodies Lab306-huFc, Lab322-huFc, and Lab323-huFc are capable of binding effectively to colon cancer cells HT55.

### 4.5 Assay on Binding of Recombinant Antibodies to Monkey GUCY2C by Flow Cytometry Assay (FACS)

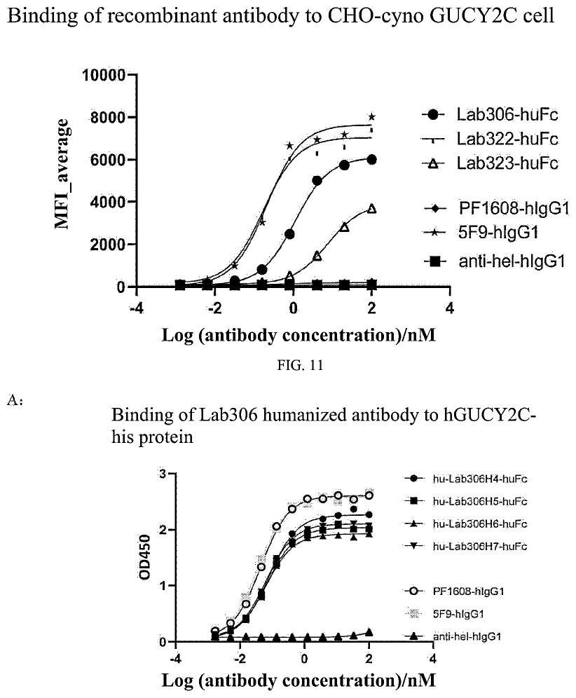

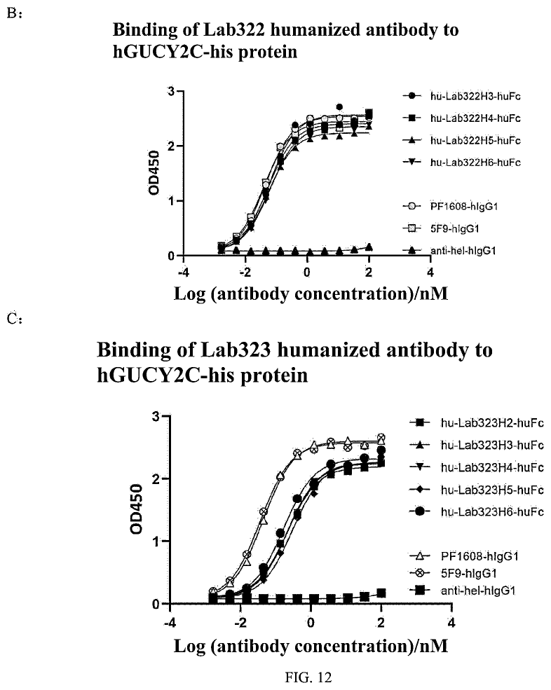

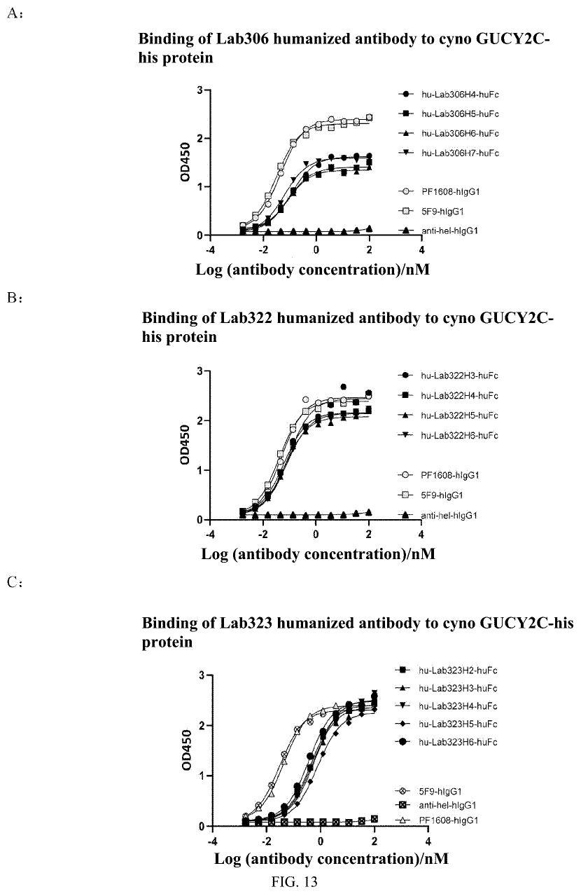

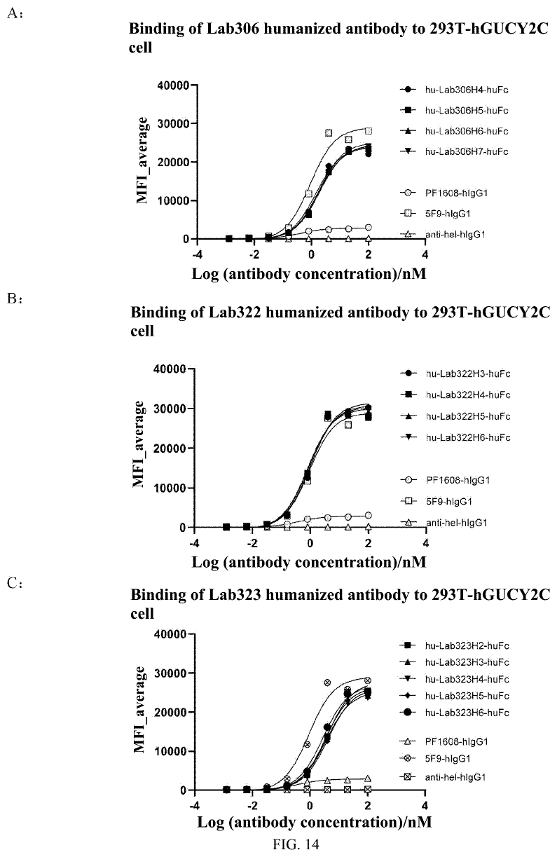

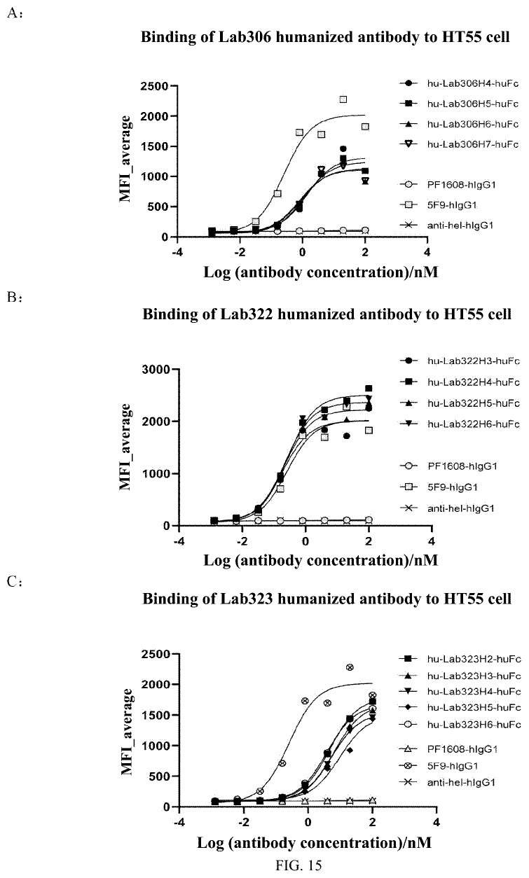

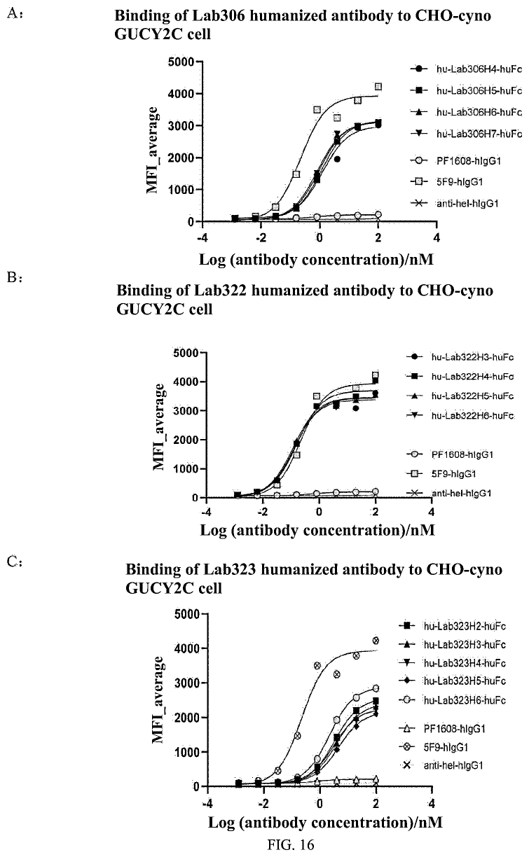

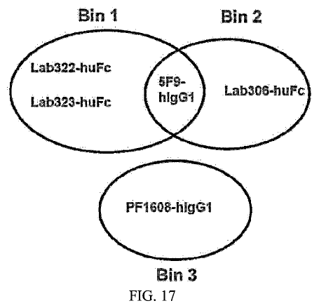

CHO-cyno GUCY2C recombinant cells were subjected to the FACS assay and data analysis according to the method described in Example 4.4. The results are shown in FIG. 11. The recombinant antibodies Lab306-huFc, Lab322-huFc, and Lab323-huFc are capable of binding effectively to FlipIn CHO-cyno GUCY2C cells.

### 4.6 Assay on Affinity of Recombinant Antibodies

The strength of antibody-antigen binding was assayed with a BIAcore 8K instrument using a Protein A capture method. First, Protein A was immobilized onto a CM4 chip (purchased from GE, BR-1005-34) using an amino coupling method. According to the instruction of the Amine Coupling Kit (purchased from GE, BR100633), NHS and EDC were mixed with HBS-EP+pH 7.4 as a mobile phase to activate the chip for about 600 s; the Protein A was diluted to 50 μg/mL with 10 mM sodium acetate (pH 4.5) and injected for 600 s, and finally, the remaining activated sites were blocked with ethanolamine. Then, the affinity of the antibody for the antigen was assayed using a multi-cycle kinetic method; in each cycle, first, the antibody to be tested was captured using the Protein A chip, and then a single concentration of antigen protein was injected. The association and dissociation processes of the antibody with the antigen protein were recorded, and finally, the chip was regenerated using Glycine pH 1.5, wherein the mobile phase was HBS-EP+(10 mM HEPES, 150 mM NaCl, 3 mM EDTA, 0.05% surfactant P20), the flow rate was 30 μL/min, the regeneration time was 30 s, and the assay temperature was 25° C. Finally, according to a 1:1 binding model, the data were analyzed, and the antibody-antigen binding kinetics parameters, including the association rate constant Ka, the dissociation rate constant Kd, the equilibrium dissociation constant KD, and the maximum binding signal Rmax, were fitted.

The association rate (Ka), dissociation rate (Kd), and binding affinity (KD) of the recombinant antibodies Lab306-huFc, Lab322-huFc, and Lab323-huFc with the human GUCY2C protein are shown in Table 5. The positive control antibody has good binding activity at the protein level, but has very weak binding at the cellular level. Lab322-huFc and Lab323-huFc, although weak in affinity, bind well to the cellular level, presumably due to differences in the conformation of the proteins and proteins at the viable cellular level.

TABLE 5
Assay on affinity of recombinant antibodies for human
GUCY2C protein by SPR (biacore)

| Recombinant | | | |
|---|---|---|---|
| antibody name | Ka (1/Ms) | Kd (1/s) | KD (M) |
| Lab306-huFc | 1.50E+05 | 2.76E−04 | 1.83E−09 |
| Lab322-huFc | 7.55E+03 | 1.17E−04 | 1.55E−08 |
| Lab323-huFc | 2.83E+03 | 2.90E−04 | 1.02E−07 |
| 5F9-hIgG1 | 1.28E+05 | 2.80E−04 | 2.19E−09 |
| PF1608-hIgG1 | 1.46E+05 | 2.35E−04 | 1.61E−09 |

Next, we measured the affinity of the recombinant antibody by another method, and the specific procedures were as follows: the binding strength of the antibody to the antigen was assayed by acquiring and analyzing the signal change of the reflection interference spectrum on the surface of the optical probe by using thin film interference technology. First, the probe was equilibrated for 300 s with HBS-T+pH 7.4 (0.02% tween) as the mobile phase. 2 μg/mL of the antibody to be tested was captured by an anti-human FC probe (purchased from ProbeLife, PL168-160003) for 240 s, and then antigen proteins with the concentration of 200 nM and diluted two-fold were injected. Kon for 300 s and Koff for 600 s. The association and dissociation processes of the antibody with the antigen protein were recorded, and finally, the chip was regenerated using Glycine pH 2.0 (purchased from GE), wherein the mobile phase was HBS-T+pH 7.4 (0.02% tween), the regeneration time was 5 s, and the assay temperature was 30° C. Finally, according to a 1:1 binding model, the data were analyzed, and the antibody-antigen binding kinetics parameters, including the association rate constant Ka, the dissociation rate constant Kd, the equilibrium dissociation constant KD, and the maximum binding signal Rmax, were fitted. The association rate (Ka), dissociation rate (Kd), and binding affinity (KD) of the recombinant antibodies with the human GUCY2C and monkey GUCY2C proteins are shown in Table 6.

TABLE 6
Assay on affinity of recombinant antibodies for human and monkey
GUCY2C proteins by Gator

| Antibody name | Antigen | koff (1/s) | kon (1/Ms) | KD (M) |
|---|---|---|---|---|
| Lab306-huFc | Human | 2.56E−04 | 2.64E+05 | 9.69E−10 |
| Lab322-huFc | GUCY2C-his | 7.86E−05 | 2.12E+05 | 3.71E−10 |
| Lab323-huFc | | 8.70E−04 | 7.14E+04 | 1.22E−08 |
| 5F9-hIgG1 | | 4.48E−04 | 3.91E+05 | 1.14E−09 |
| PF1608-hlgG1 | | 1.97E−04 | 4.00E+05 | 4.94E−10 |
| Lab306-huFc | Monkey | 3.09E−04 | 2.85E+05 | 1.08E−09 |
| Lab322-huFc | GUCY2C-his | 1.05E−04 | 2.16E+05 | 4.89E−10 |
| Lab323-huFc | | 6.00E−04 | 3.38E+04 | 1.78E−08 |
| 5F9-hIgG1 | | 4.86E−04 | 4.87E+05 | 9.98E−10 |
| PF1608-hIgG1 | | 1.38E−04 | 5.40E+05 | 2.56E−10 |

### Example 5. Humanization of Nanobody

### 5.1 Humanization Design for Nanobody

By comparing the human antibody variable region germline gene database in the IMGT database (http://imgt.cines.fr) with the Molecular Operating Environment (MOE) software, germline genes, with high homology to the nanobodies, from heavy chain variable regions were selected as templates, and CDR sequences of the nanobodies based on the Kabat naming method were separately grafted into corresponding human templates to form variable region sequences in the order of “FR1-CDR1-FR2-CDR2-FR3-CDR3-FR4”. Humanized templates of the Lab306 antibody were IGHV3-66*01 and IGHJ3*01. Humanized templates of the Lab322 antibody were IGHV3-9*01 and IGHJ3*01. Humanized templates of the Lab323 antibody were IGHV3-21*01 and IGHJ3*01. CDRs of the Vicugna pacos antibodies Lab306, Lab322, and Lab323 were respectively grafted into the human templates thereof to give humanized antibodies. The amino acid sequences of the humanized templates thereof and the humanized antibody sequences after CDR grafting are shown in Table 7.

TABLE 7
Amino acid specific sequence information of humanized template and humanized
antibody

| Name | Sequence No. | Amino acid sequence |
|---|---|---|
| IGHV3-66*01 | SEQ ID NO. 44 | EVQLVESGGGLVQPGGSLRLSCAASGFTVSSN |
| template | | YMSWVRQAPGKGLEWVSVIYSGGSTYYADS |
| | | VKGRFTISRDNSKNTLYLQMNSLRAEDTAVY |
| | | YCAR |
| IGHV3-9*01 | SEQ ID NO. 45 | EVQLVESGGGLVQPGRSLRLSCAASGFTFDDY |
| template | | AMHWVRQAPGKGLEWVSGISWNSGSIGYAD |
| | | SVKGRFTISRDNAKNSLYLQMNSLRAEDTALY |
| | | YCAKD |
| IGHV3-21*01 | SEQ ID NO. 46 | EVQLVESGGGLVKPGGSLRLSCAASGFTFSSY |
| template | | SMNWVRQAPGKGLEWVSSISSSSSYIYYADSV |
| | | KGRFTISRDNAKNSLYLQMNSLRAEDTAVYY |
| | | CAR |
| Lab306.H | SEQ ID NO. 47 | EVQLVESGGGLVQPGGSLRLSCAASGFTVSDY |
| (IGHV3-66*01 | | AIGWVRQAPGKGLEWVSCLTSGGLPKYADSV |
| Graft) | | KGRFTISRDNSKNTLYLQMNSLRAEDTAVYYC |
| | | ARSGATSRWNCPPQYDHWGQGTMVTVSS |
| Lab322.H | SEQ ID NO. 48 | EVQLVESGGGLVQPGRSLRLSCAASGFTFDYY |
| (IGHV3-9*01 | | AITWVRQAPGKGLEWVSCIQGRGTSTNYADS |
| Graft) | | VKGRFTISRDNAKNSLYLQMNSLRAEDTALY |
| | | YCAKRYVSYGRNTACGLLPESVDHWGQGTM |
| | | VTVSS |
| Lab323.H | SEQ ID NO. 49 | EVQLVESGGGLVKPGGSLRLSCAASGRTSTIH |
| (IGHV3-21*01 | | RMNWVRQAPGKGLEWVSSISRVSSTTYYADS |
| Graft) | | VKGRFTISRDNAKNSLYLQMNSLRAEDTAVY |
| | | YCAASEYEVSPGKSKYWGQGTMVTVSS |
| FR4-IGHJ3*01 | SEQ ID NO. 50 | WGQGTMVTVSS |

### 5.2. Back Mutation Design for Lab36 Humanized Antibodies

Key amino acids in the FR sequence of the Lab36 humanized antibody were back-mutated to amino acids corresponding to the Vicugna pacos antibody to ensure the original affinity. Details of the mutation points after the specific back mutation (back mutation points were numbered in a natural order) and the specific amino acid sequences are shown in Tables 8-9.

TABLE 8
Mutation point of back mutation of Lab306 humanized antibody

| Antibody name | Mutation point |
|---|---|
| hu-Lab306-H4 | V29L, S30D, V37F, |
| Graft(IGHV3-66*01) | G44E, L45R, W47G, S49I, R97A |
| hu-Lab306-H5 | A24T, V29L, S30D, V37F, G44E, L45R, W47G, |
| Graft(IGHV3-66*01) | S49I, R97A |
| hu-Lab306-H6 | V29L, S30D, V37F, G44E, L45R, W47G, S49I, |
| Graft(IGHV3-66*01) | S74A, R97A |
| hu-Lab306-H7 | V29L, S30D, V37F, G44E, L45R, W47G, S49I, |
| Graft(IGHV3-66*01) | Q81R, R97A |

Note:
Graft(IGHV3-66*01) denotes that the CDRs of the Vicugna pacos antibody are grafted onto the human germline FR sequences; and G44E denotes that G at position 44 of Graft(IGHV3-66*01) is mutated to E. and so on for others.

TABLE 9
Amino acid sequences of back mutation of Lab306 humanized antibodies

| | Sequence | |
|---|---|---|
| Antibody name | No. | Amino acid sequence |
| hu-Lab306-H4 | SEQ ID | EVQLVESGGGLVQPGGSLRLSCAASGFTLDDYAIGWF |
| | NO. 51 | RQAPGKEREGVICLTSGGLPKYADSVKGRFTISRDNSK |
| | | NTLYLQMNSLRAEDTAVYYCAASGATSRWNCPPQYD |
| | | HWGQGTMVTVSS |
| hu-Lab306-H5 | SEQ ID | EVQLVESGGGLVQPGGSLRLSCATSGFTLDDYAIGWF |
| | NO. 52 | RQAPGKEREGVICLTSGGLPKYADSVKGRFTISRDNSK |
| | | NTLYLQMNSLRAEDTAVYYCAASGATSRWNCPPQYD |
| | | HWGQGTMVTVSS |
| hu-Lab306-H6 | SEQ ID | EVQLVESGGGLVQPGGSLRLSCAASGFTLDDYAIGWF |
| | NO. 53 | RQAPGKEREGVICLTSGGLPKYADSVKGRFTISRDNA |
| | | KNTLYLQMNSLRAEDTAVYYCAASGATSRWNCPPQY |
| | | DHWGQGTMVTVSS |
| hu-Lab306-H7 | SEQ ID | EVQLVESGGGLVQPGGSLRLSCAASGFTLDDYAIGWF |
| | NO. 54 | RQAPGKEREGVICLTSGGLPKYADSVKGRFTISRDNSK |
| | | NTLYLRMNSLRAEDTAVYYCAASGATSRWNCPPQYD |
| | | HWGQGTMVTVSS |

### 5.3. Back Mutation Design for Lab322 Humanized Antibodies

Key amino acids in the FR sequence of the Lab322 humanized antibody were back-mutated to amino acids corresponding to the Vicugna pacos antibody to ensure the original affinity. Details of the mutation points after the specific back mutation (back mutation points were numbered in a natural order) and the specific amino acid sequences are shown in Tables 10-11.

TABLE 10
Mutation point of back mutation of Lab322 humanized antibody

| Antibody name | Mutation point |
|---|---|
| hu-Lab322-H3 | Graft(IGHV3-9*01) +G26V, F27L, T28N, F29L, |
| Graft(IGHV3-9*01) | V37F, G44E, L45R, W47G, N74R, N77K, L79A, |
| | K98V |
| hu-Lab322-H4 | Graft(IGHV3-9*01) +V2L, G26V, F27L, T28N, |
| Graft(IGHV3-9*01) | F29L, V37F, G44E, L45R, W47G, N74R, N77K, |
| | L79A, K98V, EIQ |
| hu-Lab322-H5 | Graft(IGHV3-9*01) +G26V, F27L, T28N, F29L, |
| Graft(IGHV3-9*01) | V37F, G44E, L45R, W47G, N74R, N77K, L79A, |
| | L93T, K98V, M124Q |
| hu-Lab322-H6 | Graft(IGHV3-9*01) +G26V, F27L, T28N, F29L, |
| Graft(IGHV3-9*01) | V37F, G44E, L45R, W47G, N74R, N77K, L79A, |
| | R87K, A88P, K98V |

Note:
Graft(IGHV3-9*01) denotes that the CDRs of the Vicugna pacos antibody are grafted onto the human germline FR sequences; and T28N denotes that T at position 28 of Graft(IGHV3-9*01) is mutated to N, and so on for others.

TABLE 11
Amino acid sequences of back mutation of Lab322 humanized antibodies

| Antibody name | Sequence No. | Amino acid sequence |
|---|---|---|
| hu-Lab322-H3 | SEQ ID | EVQLVESGGGLVQPGRSLRLSCAASVLNLDYYAIT |
| | NO. 55 | WFRQAPGKEREGVSCIQGRGTSTNYADSVKGRFT |
| | | ISRDRAKKSAYLQMNSLRAEDTALYYCAVRYVSY |
| | | GRNTACGLLPESVDHWGQGTMVTVSS |
| hu-Lab322-H4 | SEQ ID | QLQLVESGGGLVQPGRSLRLSCAASVLNLDYYAIT |
| | NO. 56 | WFRQAPGKEREGVSCIQGRGTSTNYADSVKGRFT |
| | | ISRDRAKKSAYLQMNSLRAEDTALYYCAVRYVSY |
| | | GRNTACGLLPESVDHWGQGTMVTVSS |
| hu-Lab322-H5 | SEQ ID | EVQLVESGGGLVQPGRSLRLSCAASVLNLDYYAIT |
| | NO. 57 | WFRQAPGKEREGVSCIQGRGTSTNYADSVKGRFT |
| | | ISRDRAKKSAYLQMNSLRAEDTATYYCAVRYVSY |
| | | GRNTACGLLPESVDHWGQGTQVTVSS |
| hu-Lab322-H6 | SEQ ID | EVQLVESGGGLVQPGRSLRLSCAASVLNLDYYAIT |
| | NO. 58 | WFRQAPGKEREGVSCIQGRGTSTNYADSVKGRFT |
| | | ISRDRAKKSAYLQMNSLKPEDTALYYCAVRYVSY |
| | | GRNTACGLLPESVDHWGQGTMVTVSS |

### 5.4. Back Mutation Design for Lab323 Humanized Antibodies

Key amino acids in the FR sequence of the Lab323 humanized antibody were back-mutated to amino acids corresponding to the Vicugna pacos antibody to ensure the original affinity. Details of the mutation points after the specific back mutation (back mutation points were numbered in a natural order) and the specific amino acid sequences are shown in Tables 12-13.

TABLE 12
Mutation point of back mutation of Lab323 humanized antibody

| Antibody name | Mutation point |
|---|---|
| hu-Lab323-H2 | Graft(IGHV3-21*01) |
| Graft(IGHV3-21*01) | +N35G, V37F, G44E, L45R, W47F |
| hu-Lab323-H3 | Graft(IGHV3-21*01) |
| Graft(IGHV3-21*01) | +A24S, N35G, V37F, G44E, L45R, W47F |
| hu-Lab323-H4 | Graft(IGHV3-21*01) |
| Graft(IGHV3-21*01) | +V2L, A24S, N35G, V37F, G44E, L45R, W47F |
| | +E1Q |
| hu-Lab323-H5 | Graft(IGHV3-21*01) |
| Graft(IGHV3-21*01) | +A24S, N35G, V37F, G44E, L45R, W47F, K76G |
| hu-Lab323-H6 | Graft(IGHV3-21*01) |
| Graft(IGHV3-21*01) | +A24S, N35G, V37F, G44E, L45R, W47F, L79V |

Note:
Graft(IGHV3-21*01) denotes that the CDRs of the Vicugna pacos antibody are grafted onto the human germline FR sequences; and N35G denotes that N at position 35 of Graft(IGHV3-21*01) is mutated to G, and so on for others.

TABLE 13
Amino acid sequences of back mutation of Lab323 humanized antibodies

| Antibody name | Sequence No. | Amino acid sequence |
|---|---|---|
| hu-Lab323-H2 | SEQ ID NO. 59 | EVQLVESGGGLVKPGGSLRLSCAASGRTSTIHR |
| | | MGWFRQAPGKEREFVSSISRVSSTTYYADSVKG |
| | | RFTISRDNAKNSLYLQMNSLRAEDTAVYYCAAS |
| | | EYEVSPGKSKYWGQGTMVTVSS |
| hu-Lab323-H3 | SEQ ID NO. 60 | EVQLVESGGGLVKPGGSLRLSCASSGRTSTIHR |
| | | MGWFRQAPGKEREFVSSISRVSSTTYYADSVKG |
| | | RFTISRDNAKNSLYLQMNSLRAEDTAVYYCAAS |
| | | EYEVSPGKSKYWGQGTMVTVSS |
| hu-Lab323-H4 | SEQ ID NO. 61 | QLQLVESGGGLVKPGGSLRLSCASSGRTSTIHR |
| | | MGWFRQAPGKEREFVSSISRVSSTTYYADSVKG |
| | | RFTISRDNAKNSLYLQMNSLRAEDTAVYYCAAS |
| | | EYEVSPGKSKYWGQGTMVTVSS |
| hu-Lab323-H5 | SEQ ID NO. 62 | EVQLVESGGGLVKPGGSLRLSCASSGRTSTIHR |
| | | MGWFRQAPGKEREFVSSISRVSSTTYYADSVKG |
| | | RFTISRDNAGNSLYLQMNSLRAEDTAVYYCAAS |
| | | EYEVSPGKSKYWGQGTMVTVSS |
| hu-Lab323-H6 | SEQ ID NO. 63 | EVQLVESGGGLVKPGGSLRLSCASSGRTSTIHR |
| | | MGWFRQAPGKEREFVSSISRVSSTTYYADSVKG |
| | | RFTISRDNAKNSVYLQMNSLRAEDTAVYYCAA |
| | | SEYEVSPGKSKYWGQGTMVTVSS |

### Example 6. Expression, Purification, and Identification of Humanized Nanobody

### 6.1 Expression and Purification of Humanized Nanobody

The humanized antibody variable region sequence gene was synthesized and cloned into a pTT5 vector with a human hinge region and an Fc constant region sequence to form a VHH-huFc (C220S) expression order, and a plasmid was prepared. The antibody plasmid was transiently transfected into Exp1293F cells by PEI, and after 7 days of culture, the supernatant was collected, and the antibody was purified by protein A as in Example 1.3.2.

### 6.2 Assay on Binding of Humanized Antibodies to Human GUCY2C-his Protein by Enzyme-Linked Immunosorbent Assay (ELISA)

To assay the binding activity of the humanized antibodies to human GUCY2C-his protein, a human GUCY2C-his protein was diluted with PBS to a final concentration of 2 μg/mL and then added to a 96-well ELISA plate at 50 μL/well. The plate was sealed with a plastic film and incubated at 4° C. overnight. The next day, the plate was washed 2 times with PBST, and a blocking buffer [PBS+2% (w/w) BSA] was added for blocking at room temperature for 2 h. The blocking buffer was discarded, and the plate was washed 2 times with PBST. An antibody to be tested or a control antibody diluted in a 1:3 gradient from a starting concentration of 100 nM was added at 50 μL/well. After incubation at 37° C. for 1 h, the plate was washed 3 times with PBST. A horseradish peroxidase (HRP)—labeled secondary antibody (purchased from Merck, Cat. No. AP113P) was added for incubation at 37° C. for 1 h, and the plate was washed 5 times with PBST. A TMB substrate was added at 50 L/well for incubation at room temperature for 5-10 min, and then a stop solution (1.0 M HCl) was added at 50 μL/well. OD450 nm values were read using an ELISA plate reader (Multimode Plate Reader, EnSight, purchased from Perkin Elmer).

The experimental results are shown in A-C in FIG. 12. The humanized antibodies Lab306, Lab322, and Lab323 all maintain the binding ability of the recombinant antibody and have good binding activity with the human GUCY2C protein.

### 6.3 Assay on Cross-Binding of Humanized Antibodies to Monkey GUCY2C-his Protein by Enzyme-Linked Immunosorbent Assay (ELISA)

To assay the binding activity of the humanized antibodies with the monkey GUCY2C-his protein, the monkey GUCY2C-his protein was diluted with PBS to a final concentration of 2 μg/mL and subjected to ELISA assay and data analysis according to the methods described in Example 6.2. The results are shown in A-C in FIG. 13. The humanized antibodies have cross-binding activity with the monkey GUCY2C protein, maintaining the binding ability of the recombinant antibody.

### 6.4 Assay on Binding of Humanized Antibodies to 293T-hGUCY2C Cells by Flow Cytometry Assay (FACS)

The desired cells were expanded to the logarithmic growth phase in a T-175 cell culture flask, the medium was removed by pipetting, and the cells were washed 2 times with a PBS buffer and digested with trypsin. Then a complete medium was added to stop the digestion, and the cells were blown into a single cell suspension. After counting, the cells were centrifuged, and the cell pellet was resuspended to 2×10⁶ cells/mL in an FACS buffer (PBS+2% fetal bovine serum), and added to a 96-well FACS reaction plate at 100 μL/well. The plate was centrifuged, and the supernatant was discarded. An antibody sample to be tested (diluted in a 5-fold gradient from a starting concentration of 100 nM) was added at 50 μL/well and uniformly mixed with the cells, and the mixture was incubated at 4° C. for 1 h. After the plate was centrifuged and washed 3 times with a PBS buffer, 50 μL of Alexa Fluor® 647 AffiniPure Goat Anti-Human IgG, Fcγ fragment specific-labeled secondary antibodies (purchased from Jackson, Cat. No. 109-605-098) were added to each well, and the mixture was incubated at 4° C. for 1 h. The results were tested and analyzed by FACS (FACS Canto™, purchased from BD) after the plate was centrifuged and washed 3 times with a PBS buffer and resuspended with 100 μL of PBS. Data analysis was performed by software (FlowJo) to obtain the mean fluorescence intensity (MFI) of the cells. Then, analysis was performed by software (GraphPad Prism8), data were fitted, and EC50 values were calculated. As shown in A-C in FIG. 14, PF1608-hIgG1 binds strongly to GUCY2C at the protein level, but weakly at the cellular level. The humanized antibodies of the present application are all capable of binding effectively to recombinant cells expressing human GUCY2C protein and substantially maintain the equivalent binding ability of the recombinant antibodies.

### 6.5 Assay on Binding of Humanized Antibodies to Endogenous Tumor Cells HT55 by Flow Cytometry Assay (FACS)

The preparation of the assay cells and the antibodies to be tested and the assay were performed with reference to Example 6.4. The results are shown in A-C in FIG. 15. PF1608-hIgG1 has weak binding ability to endogenous cells HT55 at the cellular level, and the humanized antibodies all have a relatively good binding activity with endogenous cells HT55, maintaining the binding ability of the corresponding recombinant antibodies before humanization. It is speculated that differences in binding at the protein and cellular levels may be related to differences in the conformation of the protein or the epitope recognized by the antibody.

### 6.6 Assay on Binding of Humanized Antibodies to CHO-Cyno GUCY2C Overexpression Cells by Flow Cytometry Assay (FACS)

The humanized antibodies were tested for binding to monkey GUCY2C at the cellular level according to the method in Example 6.4. As shown in A-C in FIG. 16, PF1608-hIgG1 has weak binding ability to overexpression cells CHO-cyno GUCY2C cells at the cellular level, and the humanized antibodies of the present application all have a relatively good cross-binding activity with CHO-cyno GUCY2C recombinant cells, maintaining the equivalent binding ability of the recombinant antibody. It is speculated that differences in binding at the protein and cellular levels may be related to differences in the conformation of the protein or the epitope recognized by the antibody.

### Example 7. Assay on Affinity of Humanized Antibodies

After the finally obtained various humanized antibodies were expressed and purified, the affinities of the humanized antibodies for the human and monkey GUCY2C proteins were separately assayed according to the method in Example 4.6. The specific affinity values are shown in Table 14 and Table 15.

TABLE 14
Assay on affinity of humanized antibodies for human
GUCY2C protein by SPR (biacore)

| Antigen name | Human GUCY2C-His | | |
|---|---|---|---|
| Antibody name | Ka(1/Ms) | Kd(1/s) | KD(M) |
| 5F9-hIgG1 | 1.40E+05 | 5.65E−04 | 4.05E−09 |
| PF1608-hIgG1 | 1.58E+05 | 2.58E−04 | 1.63E−09 |
| hu-Lab306H4-huFc | 1.11E+05 | 9.29E−04 | 8.34E−09 |
| hu-Lab306H5-huFc | 1.09E+05 | 7.86E−04 | 7.19E−09 |
| hu-Lab306H6-huFc | 1.11E+05 | 1.09E−03 | 9.87E−09 |
| hu-Lab306H7-huFc | 1.20E+05 | 1.00E−03 | 8.36E−09 |
| hu-Lab322H3-huFc | 8.24E+03 | 1.55E−04 | 1.88E−08 |
| hu-Lab322H4-huFc | 7.58E+03 | 5.52E−05 | 7.28E−09 |
| hu-Lab322H5-huFc | 8.16E+03 | 1.59E−04 | 1.95E−08 |
| hu-Lab322H6-huFc | 8.51E+03 | 1.59E−04 | 1.87E−08 |
| hu-Lab323H2-huFc | 1.01E+03 | 1.67E−04 | 1.65E−07 |
| hu-Lab323H3-huFc | 1.00E+03 | 2.74E−04 | 2.73E−07 |
| hu-Lab323H4-huFc | 1.11E+03 | 6.18E−04 | 5.58E−07 |
| hu-Lab323H5-huFc | 5.31E+06 | 1.65E+00 | 3.10E−07 |
| hu-Lab323H6-huFc | 1.36E+03 | 1.34E−04 | 9.84E−08 |

TABLE 15
Assay on affinity of humanized antibodies by Gator

| Antibody name | Antigen name | Kd (1/s) | Ka (1/Ms) | KD (M) |
|---|---|---|---|---|
| hu-Lab306H4-huFc | Human | 5.13E−04 | 2.67E+05 | 1.92E−09 |
| hu-Lab306H5-huFc | GUCY2C-His | 4.79E−04 | 2.70E+05 | 1.77E−09 |
| hu-Lab306H6-huFc | | 5.64E−04 | 2.81E+05 | 2.01E−09 |
| hu-Lab306H7-huFc | | 5.54E−04 | 2.71E+05 | 2.05E−09 |
| hu-Lab322H3-huFc | | 1.54E−04 | 2.15E+05 | 7.17E−10 |
| hu-Lab322H4-huFc | | 6.86E−05 | 2.05E+05 | 3.34E−10 |
| hu-Lab322H5-huFc | | 1.73E−04 | 2.21E+05 | 7.82E−10 |
| hu-Lab322H6-huFc | | 1.79E−04 | 2.24E+05 | 7.97E−10 |
| hu-Lab323H2-huFc | | 1.51E−03 | 5.64E+04 | 2.67E−08 |
| hu-Lab323H3-huFc | | 1.46E−03 | 5.49E+04 | 2.66E−08 |
| hu-Lab323H4-huFc | | 2.03E−03 | 6.78E+04 | 2.99E−08 |
| hu-Lab323H5-huFc | | 1.23E−03 | 4.43E+04 | 2.78E−08 |
| hu-Lab323H6-huFc | | 1.16E−03 | 5.78E+04 | 2.01E−08 |
| 5F9-hIgG1 | | 4.48E−04 | 3.91E+05 | 1.14E−09 |
| PF1608-hlgG1 | | 1.97E−04 | 4.00E+05 | 4.94E−10 |
| hu-Lab306H4-huFc | Monkey | 4.79E−04 | 3.13E+05 | 1.53E−09 |
| hu-Lab306H5-huFc | GUCY2C-His | 4.31E−04 | 2.99E+05 | 1.44E−09 |
| hu-Lab306H6-huFc | | 5.19E−04 | 3.09E+05 | 1.68E−09 |
| hu-Lab306H7-huFc | | 4.15E−04 | 3.01E+05 | 1.38E−09 |
| hu-Lab322H3-huFc | | 2.75E−04 | 2.16E+05 | 1.27E−09 |
| hu-Lab322H4-huFc | | 1.09E−04 | 2.07E+05 | 5.29E−10 |
| hu-Lab322H5-huFc | | 2.85E−04 | 2.10E+05 | 1.36E−09 |
| hu-Lab322H6-huFc | | 2.78E−04 | 2.12E+05 | 1.31E−09 |
| hu-Lab323H2-huFc | | 6.91E−04 | 3.95E+04 | 1.75E−08 |
| hu-Lab323H3-huFc | | 9.75E−04 | 2.35E+04 | 4.15E−08 |
| hu-Lab323H4-huFc | | 1.43E−03 | 2.96E+04 | 4.83E−08 |
| hu-Lab323H5-huFc | | 5.69E−04 | 2.98E+04 | 1.91E−08 |
| hu-Lab323H6-huFc | | 4.70E−04 | 3.05E+04 | 1.54E−08 |
| 5F9-hIgG1 | | 4.86E−04 | 4.87E+05 | 9.98E−10 |
| PF1608-hIgG1 | | 1.38E−04 | 5.40E+05 | 2.56E−10 |

### Example 8. Antibody-Antigen Binding Epitope Competition Experiment (Epitope Binning)

In order to identify antigen-binding sites of antibodies, antibodies were grouped using competitive ELISA. Referring to the method described in Example 4.2, ELISA plates were coated with 2 μg/mL recombinant antibodies; the human GUCY2C-his protein was subjected to a gradient dilution from 30 μg/mL, and EC80 was calculated as the antigen concentration in competitive ELISA.

The recombinant antibodies were diluted to 2 μg/mL with PBS and allowed to coat 96-well high-adsorption ELISA plates at 50 μL/well. After the plates were coated at 4° C. overnight, 250 μL of a blocking buffer (PBS containing 2% (w/v) BSA) was added for two hours of blocking at room temperature. 40 μg/mL of the antibodies to be tested were added, and then the human GUCY2C-his protein with an EC80 concentration corresponding to each of the antibodies to be tested was added for incubation for 2 h. The plates were washed 5 times with PBS, an HRP-labeled anti-His secondary antibody (purchased from Genescript, Cat. No. A00612) was then added for incubation for 1 h, and the plates were washed 5 times again. A TMB substrate was added at 50 μL/well for incubation at room temperature for 10 min, and then a stop solution (1.0 M HCl) was added at 50 μL/well. OD450 nm values were read using an ELISA plate reader (Insight, purchased from PerkinElmer), and the competition rate between the antibodies was calculated according to the OD450 nm values using a formula. The higher the value of the competition rate, the closer the epitopes to which two antibodies bind. The results are shown in FIG. 17. PF1608 does not compete with all the antibodies, and Lab322 and Lab323 antibodies are consistent with 5F9 in the epitope and compete with each other. Lab306 is close to 5F9 in the epitope and they partially compete. Accordingly, the differences in the binding ability of the antibodies at the protein and cellular levels in the previous examples are presumably related to differences in epitopes and protein conformation.

1. A nanobody or an antigen-binding fragment thereof specifically binding to GUCY2C, wherein the antibody or the antigen-binding fragment thereof comprises an HCDR1, an HCDR2, and an HCDR3, the HCDR1 comprises an HCDR1 of the VH set forth in any one of SEQ ID NOs: 14-16, 47-49, and 51-63, the HCDR2 comprises an HCDR2 of the VH set forth in any one of SEQ ID NOs: 14-16, 47-49, and 51-63, and the HCDR3 comprises an HCDR3 of the VH set forth in any one of SEQ ID NOs: 14-16, 47-49, and 51-63.

2. The nanobody or the antigen-binding fragment thereof according to claim 1, wherein the HCDR1, the HCDR2, and the HCDR3 are determined according to the Kabat numbering scheme, the Chothia numbering scheme, or the IMGT numbering scheme;
for example, the HCDR1 comprises the amino acid sequence set forth in SEQ ID NO: 17, 20, 23, 26, 29, 32, 35, 38, or 41;
the HCDR2 comprises the amino acid sequence set forth in SEQ ID NO: 18, 21, 24, 27, 30, 33, 36, 39, or 42; and
the HCDR3 comprises the amino acid sequence set forth in SEQ ID NO: 19, 22, 25, 28, 31, 34, 37, 40, or 43.

3. The nanobody or the antigen-binding fragment thereof according to claim 1, wherein an HCDR1, an HCDR2, and an HCDR3 of the VH set forth in SEQ ID NO: 14, 47, 51, 52, 53, or 54 are according to the IMGT, Kabat, or Chothia numbering scheme, and have the amino acid sequences set forth in SEQ ID NOs: 17-19, SEQ ID NOs: 26-28, or SEQ ID NOs: 35-37; an HCDR1, an HCDR2, and an HCDR3 of the VH set forth in SEQ ID NO: 15, 48, 55, 56, 57, or 58 are according to the IMGT, Kabat, or Chothia numbering scheme, and have the amino acid sequences set forth in SEQ ID NOs: 20-22, SEQ ID NOs: 29-31, or SEQ ID NOs: 38-40;
an HCDR1, an HCDR2, and an HCDR3 of the VH set forth in SEQ ID NO: 16, 49, 59, 60, 61, 62, or 63 are according to the IMGT, Kabat, or Chothia numbering scheme, and have the amino acid sequences set forth in SEQ ID NOs: 23-25, SEQ ID NOs: 32-34, or SEQ ID NOs: 41-43.

4. The nanobody or the antigen-binding fragment thereof according to claim 1, wherein the antibody or the antigen-binding fragment thereof comprises CDR sequences having at least 80% identity to the HCDR1, the HCDR2, and the HCDR3 or having 1, 2, 3, or more amino acid insertions, deletions, and/or substitutions compared with the HCDR1, the HCDR2, and the HCDR3, and preferably, the substitutions are conservative amino acid substitutions-, optionally, wherein the antibody or the antigen-binding fragment thereof comprises the VH set forth in any one of SEQ ID NOs: 14-16, 47-49, and 51-63, or a sequence having at least 80% identity to the VH set forth in any one of SEQ ID NOs: 14-16, 47-49, and 51-63 or at most 20 mutations compared with the VH set forth in any one of SEQ ID NOs: 14-16, 47-49, and 51-63: the mutation is selected from an insertion, a deletion, and/or a substitution, and preferably, the substitution is a conservative amino acid substitution.

5. (canceled)

6. The nanobody or the antigen-binding fragment thereof according to claim 4, wherein the antibody or the antigen-binding fragment thereof comprises a framework region sequence at least having a mutation, compared with a framework region of the VH set forth in SEQ ID NO: 47, selected from the group consisting of: numbered in the natural order, mutations at positions A24, V29, S30, V37, G44, L45, W47, S49, S74, Q81, and R97; preferably, the antibody or the antigen-binding fragment thereof comprises a framework region sequence at least having a mutation, compared with a framework region of the VH set forth in SEQ ID NO: 47, selected from the group consisting of: numbered in the natural order, A24T, V29L, S30D, V37F, G44E, L45R, W47G, S49I, S74A, Q81R, and R97A; preferably, at least having V29L, S30D, V37F, G44E, L45R, W47G, S49I, and R97A mutations; preferably, at least having A24T, V29L, S30D, V37F, G44E, L45R, W47G, S49I, and R97A mutations; preferably, at least having V29L, S30D, V37F, G44E, L45R, W47G, S49I, S74A, and R97A mutations; preferably, at least having V29L, S30D, V37F, G44E, L45R, W47G, S49I, Q81R, and R97A mutations;
the antibody or the antigen-binding fragment thereof comprises a framework region sequence at least having a mutation, compared with a framework region of the VH set forth in SEQ ID NO: 48, selected from the group consisting of: numbered in the natural order, mutations at positions E1, V2, G26, F27, T28, F29, V37, G44, L45, W47, N74, N77, L79, R87, A88, L93, K98, and M124; preferably, the antibody or the antigen-binding fragment thereof comprises a framework region sequence at least having a mutation, compared with a framework region of the VH set forth in SEQ ID NO: 48, selected from the group consisting of: numbered in the natural order, E1Q, V2L, G26V, F27L, T28N, F29L, V37F, G44E, L45R, W47G, N74R, N77K, L79A, R87K, A88P, L93T, K98V, and M124Q mutations;
preferably, at least having G26V, F27L, T28N, F29L, V37F, G44E, L45R, W47G, N74R, N77K, L79A, and K98V mutations; preferably, at least having V2L, G26V, F27L, T28N, F29L, V37F, G44E, L45R, W47G, N74R, N77K, L79A, K98V, and E1Q mutations; preferably, at least having G26V, F27L, T28N, F29L, V37F, G44E, L45R, W47G, N74R, N77K, L79A, L93T, K98V, and M124Q mutations; preferably, at least having G26V, F27L, T28N, F29L, V37F, G44E, L45R, W47G, N74R, N77K, L79A, R87K, A88P, and K98V mutations;
or the antibody or the antigen-binding fragment thereof comprises a framework region sequence at least having a mutation, compared with a framework region of the VH set forth in SEQ ID NO: 49, selected from the group consisting of: numbered in the natural order, mutations at position E1, V2, A24, N35, V37, G44, L45, W47, K76, or L79; preferably, the antibody or the antigen-binding fragment thereof comprises a framework region sequence at least having a mutation, compared with a framework region of the VH set forth in SEQ ID NO: 49, selected from the group consisting of: numbered in the natural order, E1Q, V2L, A24S, N35G, V37F, G44E, L45R, W47F, K76G, and L79V; preferably, at least having N35G, V37F, G44E, L45R, and W47F mutations; more preferably, at least having A24S, N35G, V37F, G44E, L45R, and W47F mutations; preferably, at least having V2L, A24S, N35G, V37F, G44E, L45R, W47F, and E1Q mutations; preferably, at least having A24S, N35G, V37F, G44E, L45R, W47F, and K76G mutations; preferably, at least having A24S, N35G, V37F, G44E, L45R, W47F, and L79V mutations.

7. The nanobody or the antigen-binding fragment thereof according to claim 1, wherein the antibody or the antigen-binding fragment thereof specifically binds to both human and monkey GUCY2C proteins; preferably, the antibody or the antigen-binding fragment thereof binds to human and monkey GUCY2C proteins with a KD superior to 6.00E-7 M optionally, wherein the antibody or the antigen-binding fragment thereof is: (1) a chimeric antibody or a fragment thereof; (2) a humanized antibody or a fragment thereof; or (3) a fully human antibody or a fragment thereof.

8. (canceled)

9. The nanobody or the antigen-binding fragment thereof according to claim 1, wherein the nanobody or the antigen-binding fragment thereof comprises or does not comprise an antibody heavy chain constant region; optionally, the antibody heavy chain constant region is selected from human, Vicugna pacos, mouse, rat, rabbit, or sheep; optionally, the antibody heavy chain constant region is selected from IgG, IgM, IgA, IgE, or IgD, and the IgG is selected from IgG1, IgG2, IgG3, or IgG4; optionally, the heavy chain constant region is selected from an Fc region, a CH3 region, or an intact heavy chain constant region; preferably, the heavy chain constant region is a human Fc region; preferably, the nanobody or the antigen-binding fragment thereof is a heavy chain antibody, optionally, wherein the nanobody or the antigen-binding fragment thereof is further conjugated to a therapeutic agent or a tracer; preferably, the therapeutic agent is selected from a drug, a toxin, a radioisotope, a chemotherapeutic agent, or an immunomodulator, and preferably, the tracer is selected from a radiocontrast medium, a paramagnetic ion, a metal, a fluorescent label, a chemiluminescent label, an ultrasound contrast agent, and a photosensitizer.

10. (canceled)

11. A multispecific molecule, wherein the multispecific molecule comprises the nanobody or the antigen-binding fragment thereof according to claim 1; preferably, the multispecific molecule further comprises a nanobody or an antigen-binding fragment thereof specifically binding to an antigen other than GUCY2C or binding to a GUCY2C epitope different from the nanobody or the antigen-binding fragment thereof according to claim 1, optionally, wherein the antigen other than GUCY2C is an antigen on the surface of a T cell, a B cell, a natural killer cell, a dendritic cell, a macrophage, a monocyte, or a neutrophil; preferably, the antigen other than GUCY2C is selected from: CD96, PD-1, PD-L1, PD-L2, OX40, OX40L, LAG-3, TIM3, VISTA, CD3, CD3γ, CD3δ, CD3ε, CD3ζ, CD27, CD28, CD28H, CD16, CD16A, CD32B, VEGF, NKG2D, NKp30, NKp46, NKp44, CD19, CD20, CD40, CD47, 4-1BB, ICOS, OX40, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WTi, NY-ESO-1, MAGE3, ASGPRI, TGFβ-trap, IL-2, IL-15, IL-21, IL-18, or CDH16; preferably, the multispecific molecule is bispecific, trispecific, or tetraspecific, and more preferably, the multispecific molecule is divalent, tetravalent, or hexavalent, optionally wherein the multispecific molecule is a tandem scFv, a bifunctional antibody (Db), a single chain bifunctional antibody (scDb), a dual affinity retargeting (DART) antibody, a F(ab′)₂, a dual variable domain (DVD) antibody, a knobs-into-holes (KiH) antibody, a dock-and-lock (DNL) antibody, a chemically cross-linked antibody, a heteropolymeric nanobody, or a heteroconjugate antibody.

12. (canceled)

13. (canceled)

14. A chimeric antigen receptor (CAR), wherein the chimeric antigen receptor at least comprises an extracellular antigen-binding domain, a transmembrane domain, and an intracellular signaling domain; the extracellular antigen-binding domain comprises the nanobody or the antigen-binding fragment thereof according to claim 1.

15. An immune effector cell, wherein the immune effector cell expresses the chimeric antigen receptor according to claim 14 or comprises a nucleic acid fragment encoding the chimeric antigen receptor according to claim 14; preferably, the immune effector cell is selected from a T cell, an NK cell, an NKT cell, a DNT cell, a monocyte, a macrophage, a dendritic cell, or a mast cell, and preferably, the T cell is selected from a cytotoxic T cell, a regulatory T cell, or a helper T cell; preferably, the immune effector cell is an auto-immune effector cell or an allogeneic immune effector cell.

16. An isolated nucleic acid fragment, wherein the nucleic acid fragment encodes the nanobody or the antigen-binding fragment thereof according to claim 1.

17. A vector, wherein the vector comprises the nucleic acid fragment according to claim 16.

18. A host cell, wherein the host cell comprises the vector according to claim 17;
preferably, the cell is a prokaryotic cell or a eukaryotic cell, such as a bacterium (e.g., E. coli), a fungus (e.g., yeast), an insect cell, or a mammalian cell (e.g., a CHO cell line or a 293T cell line).

19. A method for preparing the nanobody or the antigen-binding fragment thereof according to claim 1, wherein the method comprises culturing a host cell, and isolating a nanobody or an antigen-binding fragment thereof expressed by the cell, or isolating a multispecific molecule expressed by the cell, wherein the host cell comprises a vector comprising a nucleic acid fragment encoding the nanobody or the antigen-binding fragment thereof according to claim 1.

20. A method for preparing an immune effector cell, wherein the method comprises introducing a nucleic acid fragment encoding the CAR according to claim 14 into the immune effector cell; optionally, the method further comprises initiating expression of the CAR according to claim 14 in the immune effector cell, preferably, the immune effector cell is selected from a T cell, an NK cell, an NKT cell, a DNT cell, a monocyte, a macrophage, a dendritic cell, or a mast cell, and preferably, the T cell is selected from a cytotoxic T cell, a regulatory T cell, or a helper T cell; preferably, the immune effector cell is an auto-immune effector cell or an allogeneic immune effector cell.

21. A pharmaceutical composition, wherein the pharmaceutical composition comprises the nanobody or the antigen-binding fragment thereof according to claim 1, optionally, the pharmaceutical composition further comprises a pharmaceutically acceptable carrier, diluent, or adjuvant.

22. (canceled)

23. A method for preventing and/or treating a tumor, wherein the method comprises administering to a patient in need thereof an effective amount of the nanobody or the antigen-binding fragment thereof according to claim 1, wherein the tumor is selected from colorectal cancer, gastric cancer, small intestine cancer, esophageal cancer, pancreatic cancer, lung cancer, soft tissue sarcoma, and neuroendocrine tumor.

24. (canceled)

25. A kit, wherein the kit comprises the nanobody or the antigen-binding fragment thereof according to claim 1.

26. A method for detecting GUCY2C expression, comprising contacting a sample to be tested with the nanobody or the antigen-binding fragment thereof according to claim 1 in a condition allowing formation of a complex by the nanobody or the antigen-binding fragment thereof according to claim 1 and GUCY2C.

27. A method for inhibiting the proliferation or migration of a cell expressing GUCY2C in vitro, comprising contacting the cell with the nanobody or the antigen-binding fragment thereof according to claim 1 in a condition allowing formation of a complex by the nanobody or the antigen-binding fragment thereof according to claim 1 and GUCY2C.


SEQUENCE LISTING (63 sequences)

SEQ ID NO: 1 - protein, 413 aa, synthetic construct
SQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQHHHHHH

SEQ ID NO: 2 - protein, 413 aa, synthetic construct
SQVSQNCHNGSYEISVLMMDNSAFAEPLENVEDAVNEGLEIVRGRLQNAGLNVTVNASFMYSDGLIHNSGDCRSSTCEGLDLLRKISNAKRMGCVLMGPSCTYSTFQMYLDTELSYPMISAGSFGLSCDYKETLTRLMSPARKLTYFLVNFWKTNDLPFKTYSWSTSYVYKNGTESEDCFWYLNALEASVSYFSHELSFKLVLRQDKEFQDILMDHNRKSNVIVMCGDPEFLYKLKGDRAVAEDIVIILVDLFNDQYFEDNVTAPDYMKNVLVLTQSPGNSLLNSSFSRNLSPTKRDFALAYLNGILLFGHMLKTFLENGENITTPKFAHAFRNLTFEGYDGPVTLDDWGDVDSTMVLLYTSVDTKKYKVLLTYDTHVNQTNPVDMSPTFTWKNSKLPNDITDRGPQHHHHHH

SEQ ID NO: 3 - protein, 420 aa, synthetic construct
VFWASQVRQNCRNGSYEISVLMMDNSAYKEPMQNLREAVEEGLDIVRKRLREADLNVTVNATFIYSDGLIHKSGDCRSSTCEGLDLLREITRDHKMGCALMGPSCTYSTFQMYLDTELNYPMISAGSYGLSCDYKETLTRILPPARKLMYFLVDFWKVNNASFKPFSWNSSYVYKNGSEPEDCFWYLNALEAGVSYFSEVLNFKDVLRRSEQFQEILTGHNRKSNVIVMCGTPESFYDVKGDLQVAEDTVVILVDLFSNHYFEENTTAPEYMDNVLVLTLPSEQSTSNTSVAERFSSGRSDFSLAYLEGTLLFGHMLQTFLENGENVTGPKFARAFRNLTFQGFAGPVTLDDSGDIDNIMSLLYVSLDTRKYKVLMKYDTHKNKTIPVAENPNFIWKNHKLPNDVPGLGPQILMHHHHHH

SEQ ID NO: 4 - protein, 449 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 452 aa, synthetic construct
EVQLVESGGGLVQPGGSLRLSCAASGFTFSSYWMHWVRQAPGKGLEWIGEIKPSNELTNVHEKFKDRFTISVDKAKNSAYLQMNSLRAEDTAVYYCTRTITTTEGYWFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 217 aa, synthetic construct
DIQLTQSPSSLSASVGDRVTITCRASESVDYYGSSLLQWYQQKPGKAPKLLIYAASKLASGVPSRFSGSGSGTDFTLTISSLQPEDFATYYCQQTRKAYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 18 bp, synthetic construct
  source 1..18: mol_type = other DNA
cttggtggtcctggctgc

SEQ ID NO: 10 - DNA, 23 bp, synthetic construct
  source 1..23: mol_type = other DNA
ggtacgtgctgttgaactgttcc

SEQ ID NO: 11 - DNA, 47 bp, synthetic construct
  source 1..47: mol_type = other DNA
catgccatgactgtggcccaggcggcccagktgcagctcgtggagtc

SEQ ID NO: 12 - DNA, 51 bp, synthetic construct
  source 1..51: mol_type = other DNA
catgccatgactcgcggccggcctggccatgggggtcttcgctgtggtgcg

SEQ ID NO: 13 - DNA, 52 bp, synthetic construct
  source 1..52: mol_type = other DNA
catgccatgactcgcggccggcctggccgtcttgtggttttggtgtcttggg

SEQ ID NO: 14 - protein, 123 aa, synthetic construct
QLQLVESGGGLVQPGGSLRLSCVTSGFTLDDYAIGWFRQAPGKEREGVICLTSGGLPKYADSVKGRFTISRDNAKNTVYLRMDSLKPDDTAVYYCAASGATSRWNCPPQYDHWGQGTQVTVSS

SEQ ID NO: 15 - protein, 129 aa, synthetic construct
QLQLVESGGGLVQPGGSLRLSCAASVLNLDYYAITWFRQAPGKEREGVSCIQGRGTSTNYADSVKGRFTISRDRAKKTAYLQMNSLKPEDTATYYCAVRYVSYGRNTACGLLPESVDHWGQGTQVTVSS

SEQ ID NO: 16 - protein, 121 aa, synthetic construct
QLQLVESGGGLVQPGGSLRLSCVSSGRTSTIHRMGWFRQAPGKERDFVASISRVSSTTYYADSVKGRFTISRDNAGNTVYLQMNSLKPEDTAVYYCAASEYEVSPGKSKYWGQGTQVTVSS

SEQ ID NO: 17 - protein, 8 aa, synthetic construct
GFTLDDYA

SEQ ID NO: 18 - protein, 7 aa, synthetic construct
LTSGGLP

SEQ ID NO: 19 - protein, 17 aa, synthetic construct
AASGATSRWNCPPQYDH

SEQ ID NO: 20 - protein, 8 aa, synthetic construct
VLNLDYYA

SEQ ID NO: 21 - protein, 8 aa, synthetic construct
IQGRGTST

SEQ ID NO: 22 - protein, 22 aa, synthetic construct
AVRYVSYGRNTACGLLPESVDH

SEQ ID NO: 23 - protein, 8 aa, synthetic construct
GRTSTIHR

SEQ ID NO: 24 - protein, 8 aa, synthetic construct
ISRVSSTT

SEQ ID NO: 25 - protein, 14 aa, synthetic construct
AASEYEVSPGKSKY

SEQ ID NO: 26 - protein, 5 aa, synthetic construct
DYAIG

SEQ ID NO: 27 - protein, 16 aa, synthetic construct
CLTSGGLPKYADSVKG

SEQ ID NO: 28 - protein, 15 aa, synthetic construct
SGATSRWNCPPQYDH

SEQ ID NO: 29 - protein, 5 aa, synthetic construct
YYAIT

SEQ ID NO: 30 - protein, 17 aa, synthetic construct
CIQGRGTSTNYADSVKG

SEQ ID NO: 31 - protein, 20 aa, synthetic construct
RYVSYGRNTACGLLPESVDH

SEQ ID NO: 32 - protein, 5 aa, synthetic construct
IHRMG

SEQ ID NO: 33 - protein, 17 aa, synthetic construct
SISRVSSTTYYADSVKG

SEQ ID NO: 34 - protein, 12 aa, synthetic construct
SEYEVSPGKSKY

SEQ ID NO: 35 - protein, 7 aa, synthetic construct
GFTLDDY

SEQ ID NO: 36 - protein, 5 aa, synthetic construct
TSGGL

SEQ ID NO: 37 - protein, 15 aa, synthetic construct
SGATSRWNCPPQYDH

SEQ ID NO: 38 - protein, 7 aa, synthetic construct
VLNLDYY

SEQ ID NO: 39 - protein, 6 aa, synthetic construct
QGRGTS

SEQ ID NO: 40 - protein, 20 aa, synthetic construct
RYVSYGRNTACGLLPESVDH

SEQ ID NO: 41 - protein, 7 aa, synthetic construct
GRTSTIH

SEQ ID NO: 42 - protein, 6 aa, synthetic construct
SRVSST

SEQ ID NO: 43 - protein, 12 aa, synthetic construct
SEYEVSPGKSKY

SEQ ID NO: 44 - protein, 97 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 45 - protein, 99 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 46 - protein, 98 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 47 - protein, 123 aa, synthetic construct
EVQLVESGGGLVQPGGSLRLSCAASGFTVSDYAIGWVRQAPGKGLEWVSCLTSGGLPKYADSVKGRFTISRDNSKNTLYLQMNSLRAEDTAVYYCARSGATSRWNCPPQYDHWGQGTMVTVSS

SEQ ID NO: 48 - protein, 129 aa, synthetic construct
EVQLVESGGGLVQPGRSLRLSCAASGFTFDYYAITWVRQAPGKGLEWVSCIQGRGTSTNYADSVKGRFTISRDNAKNSLYLQMNSLRAEDTALYYCAKRYVSYGRNTACGLLPESVDHWGQGTMVTVSS

SEQ ID NO: 49 - protein, 121 aa, synthetic construct
EVQLVESGGGLVKPGGSLRLSCAASGRTSTIHRMNWVRQAPGKGLEWVSSISRVSSTTYYADSVKGRFTISRDNAKNSLYLQMNSLRAEDTAVYYCAASEYEVSPGKSKYWGQGTMVTVSS

SEQ ID NO: 50 - protein, 11 aa, synthetic construct
WGQGTMVTVSS

SEQ ID NO: 51 - protein, 123 aa, synthetic construct
EVQLVESGGGLVQPGGSLRLSCAASGFTLDDYAIGWFRQAPGKEREGVICLTSGGLPKYADSVKGRFTISRDNSKNTLYLQMNSLRAEDTAVYYCAASGATSRWNCPPQYDHWGQGTMVTVSS

SEQ ID NO: 52 - protein, 123 aa, synthetic construct
EVQLVESGGGLVQPGGSLRLSCATSGFTLDDYAIGWFRQAPGKEREGVICLTSGGLPKYADSVKGRFTISRDNSKNTLYLQMNSLRAEDTAVYYCAASGATSRWNCPPQYDHWGQGTMVTVSS

SEQ ID NO: 53 - protein, 123 aa, synthetic construct
EVQLVESGGGLVQPGGSLRLSCAASGFTLDDYAIGWFRQAPGKEREGVICLTSGGLPKYADSVKGRFTISRDNAKNTLYLQMNSLRAEDTAVYYCAASGATSRWNCPPQYDHWGQGTMVTVSS

SEQ ID NO: 54 - protein, 123 aa, synthetic construct
EVQLVESGGGLVQPGGSLRLSCAASGFTLDDYAIGWFRQAPGKEREGVICLTSGGLPKYADSVKGRFTISRDNSKNTLYLRMNSLRAEDTAVYYCAASGATSRWNCPPQYDHWGQGTMVTVSS

SEQ ID NO: 55 - protein, 129 aa, synthetic construct
EVQLVESGGGLVQPGRSLRLSCAASVLNLDYYAITWFRQAPGKEREGVSCIQGRGTSTNYADSVKGRFTISRDRAKKSAYLQMNSLRAEDTALYYCAVRYVSYGRNTACGLLPESVDHWGQGTMVTVSS

SEQ ID NO: 56 - protein, 129 aa, synthetic construct
QLQLVESGGGLVQPGRSLRLSCAASVLNLDYYAITWFRQAPGKEREGVSCIQGRGTSTNYADSVKGRFTISRDRAKKSAYLQMNSLRAEDTALYYCAVRYVSYGRNTACGLLPESVDHWGQGTMVTVSS

SEQ ID NO: 57 - protein, 129 aa, synthetic construct
EVQLVESGGGLVQPGRSLRLSCAASVLNLDYYAITWFRQAPGKEREGVSCIQGRGTSTNYADSVKGRFTISRDRAKKSAYLQMNSLRAEDTATYYCAVRYVSYGRNTACGLLPESVDHWGQGTQVTVSS

SEQ ID NO: 58 - protein, 129 aa, synthetic construct
EVQLVESGGGLVQPGRSLRLSCAASVLNLDYYAITWFRQAPGKEREGVSCIQGRGTSTNYADSVKGRFTISRDRAKKSAYLQMNSLKPEDTALYYCAVRYVSYGRNTACGLLPESVDHWGQGTMVTVSS

SEQ ID NO: 59 - protein, 121 aa, synthetic construct
EVQLVESGGGLVKPGGSLRLSCAASGRTSTIHRMGWFRQAPGKEREFVSSISRVSSTTYYADSVKGRFTISRDNAKNSLYLQMNSLRAEDTAVYYCAASEYEVSPGKSKYWGQGTMVTVSS

SEQ ID NO: 60 - protein, 121 aa, synthetic construct
EVQLVESGGGLVKPGGSLRLSCASSGRTSTIHRMGWFRQAPGKEREFVSSISRVSSTTYYADSVKGRFTISRDNAKNSLYLQMNSLRAEDTAVYYCAASEYEVSPGKSKYWGQGTMVTVSS

SEQ ID NO: 61 - protein, 121 aa, synthetic construct
QLQLVESGGGLVKPGGSLRLSCASSGRTSTIHRMGWFRQAPGKEREFVSSISRVSSTTYYADSVKGRFTISRDNAKNSLYLQMNSLRAEDTAVYYCAASEYEVSPGKSKYWGQGTMVTVSS

SEQ ID NO: 62 - protein, 121 aa, synthetic construct
EVQLVESGGGLVKPGGSLRLSCASSGRTSTIHRMGWFRQAPGKEREFVSSISRVSSTTYYADSVKGRFTISRDNAGNSLYLQMNSLRAEDTAVYYCAASEYEVSPGKSKYWGQGTMVTVSS

SEQ ID NO: 63 - protein, 121 aa, synthetic construct; identical to SEQ ID NO: 62